(19) United States
(12) Patent Application Publication
(10) Pub. No.: US 2025/0241577 A1
(43) Pub. Date: Jul. 31, 2025

(54) Wearable Band Structure With Flexible Printed Circuit Strain-Relief Integration, And Systems And Methods Of Use Thereof

(71) Applicant: Meta Platforms Technologies, LLC, Menlo Park, CA (US)

(72) Inventors: Marc-Aurelien Vivant, Brooklyn, NY (US); Melissa Zucker, San Francisco, CA (US); Nicholas Alan Fraser, Hayward, CA (US)

(21) Appl. No.: 19/000,546

(22) Filed: Dec. 23, 2024

Related U.S. Application Data
(60) Provisional application No. 63/626,511, filed on Jan. 29, 2024.

(51) Int. Cl. A61B 5/256; A44C 5/00; A61B 5/00; G04G 21/02; H05K 1/02

(52) U.S. Cl. CPC A61B 5/256; A44C 5/0015; A61B 5/6803; A61B 5/681; H05K 1/0277; A61B 2562/043; A61B 2562/164; A61B 2562/166; G04G 21/025

(57) ABSTRACT

A wearable band structure that includes a flexible printed circuit layer and a strain-relief layer is described. The flexible printed circuit layer is within the wearable structure and configured to couple with one or more biopotential signal-processing components along a coupling length of the flexible printed circuit layer. The strain-relief layer is coupled with a portion of the flexible printed circuit layer such that the strain-relief layer spans the coupling length of the flexible printed circuit layer. A strain applied to the wearable structure is relieved by the strain-relief layer such that the strain is not transferred to the one or more biopotential signal-processing components.

### CROSS-REFERENCE TO RELATED APPLICATIONS

This application claims priority to U.S. Prov. Patent App. No. 63/626,511, filed Jan. 29, 2024, entitled “A Wearable Band Structure with Flexible Printed Circuit Strain-Relief Integration, and Systems and Methods of Use Thereof,” which is hereby fully incorporated by reference in its entirety.

### TECHNICAL FIELD

The present disclosure relates generally to wearable electronic devices (e.g., wrist-worn wearable devices) and more specifically to wearable electronic devices having a band portion including flexible printed circuit(s) and electronics components (e.g., components that detect and/or at least partially process biopotential signals of a user).

### BACKGROUND

Traditional conductive materials are inherently rigid, posing challenges for their application in wearable devices (e.g., wrist-wearable devices) that undergo continuous strain and stress during usage (e.g., tugging, pulling, and stretching of a wearable band). While flexible electronics, such as flexible printed circuit assemblies, are suitable for wearable devices, their current implementation in wearable devices have shortened lifespans due to the constant strain and stress. As such, there is a demand for innovations aimed at enhancing the strength and durability of flexible printed circuit assemblies when integrated into wearable devices.

Existing technology in this space is limited. Current solutions are often bulky and need to sacrifice on performance and reliability. As a result, the solutions need to be compact and fit seamlessly within the current form factor of the wrist-wearable devices without reducing the flexibility of the system.

As such, there is a need to address one or more of the above-identified challenges. A brief summary of solutions to the issues noted above are described below.

### SUMMARY

The methods, systems, and devices disclosed herein, overcome the above challenges by combining the right balance of support, flexibility, and electronic integration into wearable devices.

To enhance the strength and durability of flexible printed circuit assemblies of a wearable device, one solution is to optimize the stack-up of a wearable band of the wearable device. For example, in one aspect, the wearable band can include a strain-relief layer that aligns flexible printed circuit assemblies along a neutral axis to offload the stress, thereby limiting the bend radius and further protecting electronics components. In particular, the strain-relief layer (e.g., Vectran, Kevlar, or other polymers) can be integrated into flexible printed circuit assemblies to improve their rigidity. Moreover, the strain-relief layer can be configured to couple with flexible printed circuit assemblies such that a strain and stress caused by tugging, pulling, and stretching on the wearable band can be transferred to the strain-relief layer before reaching out to flexible printed circuit assemblies. Furthermore, integrating the strain-relief layer into flexible printed circuit assemblies is compatible with the current form factor of the wearable band and can facilitate flexibility of resizing. In addition, a modular approach can be adopted to enable alternative configurations under the same principle, where layers in the stack-up of the wearable band are laminated or formed externally to conventional flexible printed circuit assemblies.

One example of a wearable band structure is described herein. This example wearable band structure includes a flexible printed circuit layer and a strain-relief layer. The flexible printed circuit layer is within the wearable band structure and configured to couple with one or more biopotential signal-processing components along a coupling length of the flexible printed circuit layer. The strain-relief layer is coupled with a portion of the flexible printed circuit layer such that the strain-relief layer spans the coupling length of the flexible printed circuit layer. A strain applied to the wearable structure is relieved by the strain-relief layer such that the strain is not transferred to the one or more biopotential signal-processing components.

The features and advantages described in the specification are not necessarily all inclusive and, in particular, certain additional features and advantages will be apparent to one of ordinary skill in the art in view of the drawings, specification, and claims. Moreover, it should be noted that the language used in the specification has been principally selected for readability and instructional purposes.

Having summarized the above example aspects, a brief description of the drawings will now be presented.

### BRIEF DESCRIPTION OF THE DRAWINGS

For a better understanding of the various described embodiments, reference should be made to the Detailed Description below, in conjunction with the following drawings in which like reference numerals refer to corresponding parts throughout the figures.

FIGS. 1A-1C illustrate an example wearable device for sensing biopotential signals at a user's skin, in accordance with some embodiments.

FIG. 2 illustrates a cross-sectional view of an example wearable device, in accordance with some embodiments.

FIGS. 3A-3B illustrate different views along a lengthwise portion of a band portion, in accordance with some embodiments.

FIG. 4 illustrates cross-sectional views of example receiving structures, in accordance with some embodiments.

FIGS. 5A-5D illustrate a first example embodiment of a band portion of a wearable device, in accordance with some embodiments.

FIGS. 6A-6D illustrate a second example embodiment of a band portion of a wearable device, in accordance with some embodiments.

FIG. 7 illustrates different flex stack-up embodiments, in accordance with some embodiments.

FIG. 8 illustrates one or more layers of a third example band portion of a wearable device, in accordance with some embodiments.

FIGS. 9A-9B illustrate cross-sectional views of integrated strain-relief layers, in accordance with some embodiments.

FIG. 10A illustrates an analog front end (AFE) offloaded example band portion of a wearable device, in accordance with some embodiments.

FIG. 10B illustrates AFEs removed from a flexible printed circuit layer, in accordance with some embodiments.

FIG. 11 illustrates an alternate band portion of a wearable device, in accordance with some embodiments.

FIG. 12 illustrates a flow diagram of an example method of manufacturing a wearable structure of a wearable device, in accordance with some embodiments.

FIG. 13 illustrates an example artificial-reality system, in accordance with some embodiments.

FIGS. 14A-14B illustrate an example wrist-wearable device, in accordance with some embodiments.

In accordance with common practice, the various features illustrated in the drawings may not be drawn to scale. Accordingly, the dimensions of the various features may be arbitrarily expanded or reduced for clarity. In addition, some of the drawings may not depict all of the components of a given system, method, or device. Finally, like reference numerals may be used to denote like features throughout the specification and figures.

### DETAILED DESCRIPTION

Numerous details are described herein to provide a thorough understanding of the example embodiments illustrated in the accompanying drawings. However, some embodiments may be practiced without many of the specific details, and the scope of the claims is only limited by those features and aspects specifically recited in the claims. Furthermore, well-known processes, components, and materials have not necessarily been described in exhaustive detail so as to avoid obscuring pertinent aspects of the embodiments described herein.

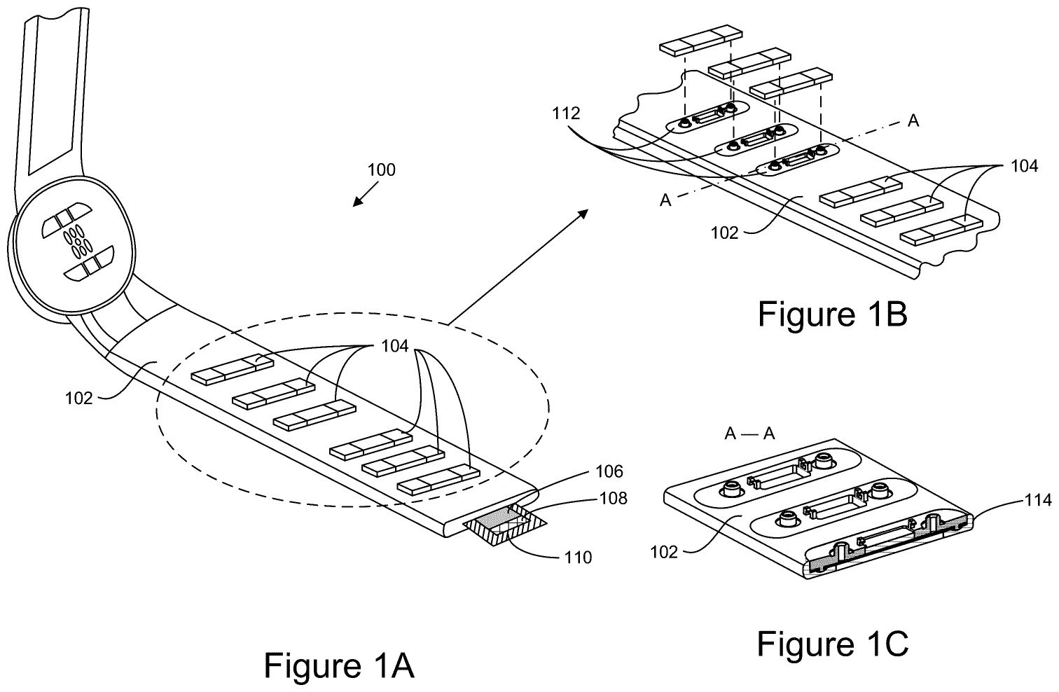

FIGS. 1A-1C illustrate an example wearable device 100 for sensing biopotential signals at a user's skin, in accordance with some embodiments. FIG. 1A shows a perspective view of the wearable device 100, FIG. 1B shows an exploded view of the wearable device 100, and FIG. 1C shows a perspective cross-sectional view of the wearable device 100. The wearable device 100 can be a wrist-wearable device (e.g., a smart watch, a band) that is configured to be worn on a wrist of the user (e.g., a wearer). In some embodiments, the wearable device 100 can be an armband, headband, chest strap, and/or other device that can be worn on the user's body.

As shown in FIG. 1A, the wearable device 100 includes a band portion 102 and one or more biopotential-signal sensing structures 104. The biopotential-signal sensing structures 104 are distributed along a length of the band portion 102 and configured to contact distinct portions of a skin of the user. The band portion 102 includes one or more interior layers such as a flexible printed circuit (FPC) layer 106, a strain-relief layer 108, and an overmolded metal layer 110. In some embodiments, the one or more interiors layers are coupled via adhesive layers. For example, in some embodiments, one or more adhesive layers (not shown in FIG. 1A) couple the strain-relief layer 108 with one or more other interior layers.

Turning to FIG. 1B, in some embodiments, the biopotential-signal sensing structures 104 are configured to couple with receiving structures 112. The receiving structures 112 can include exposed portions that are configured to affix the biopotential-signal sensing structures 104 to the wearable device 100 and communicatively coupled the biopotential-signal sensing structures 104 with other components of the wearable device 100, such as the FPC layer 106 as shown in FIG. 1A.

The perspective cross-sectional view 114 of the wearable device 100, as shown in FIG. 1C, illustrates a cut through A-A along a widthwise portion of the band portion 102. The perspective cross-sectional view 114 of the wearable device 100 show one or more example interior layers of the wearable device 100. For example, the one or more interior layers include the FPC layer 106, strain-relief layer 108, and overmolded metal layer 110, as shown in FIG. 1A.

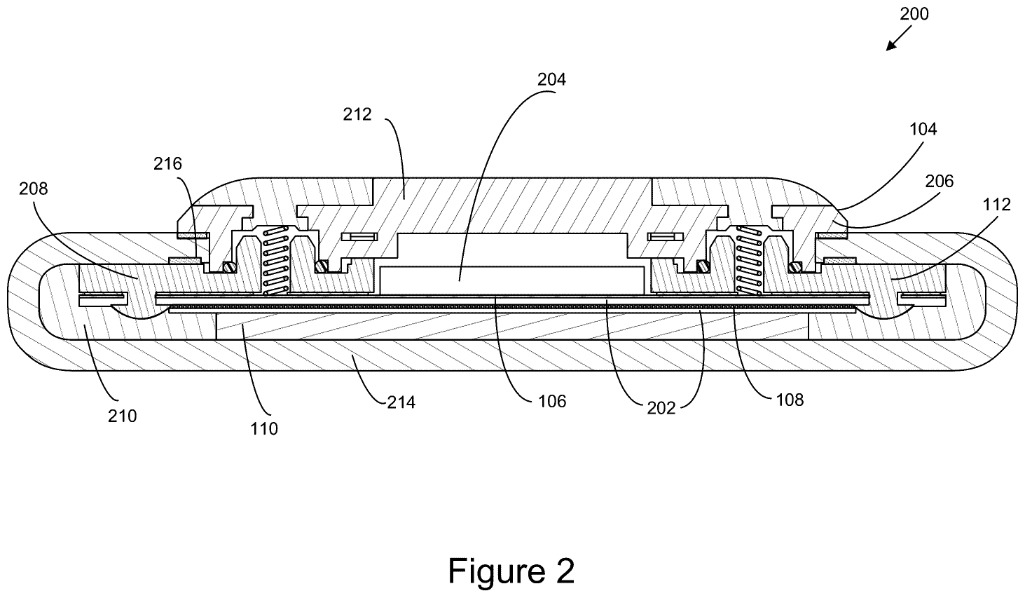

FIG. 2 illustrates a cross-sectional view of the example wearable device 100, in accordance with some embodiments. The wearable device 100 includes an FPC 106, a strain-relief layer 108, adhesive layers 202, an overmolded metal layer 110, an analog front end (AFE) 204, a coupling portion 206 of biopotential-signal sensing structure 104, an edge support portion 216, and a receiving structure 112. The wearable device 100 further includes a band overmold 210, a low pressure molding 212, a textile 214, and other components that are not labeled in FIG. 2.

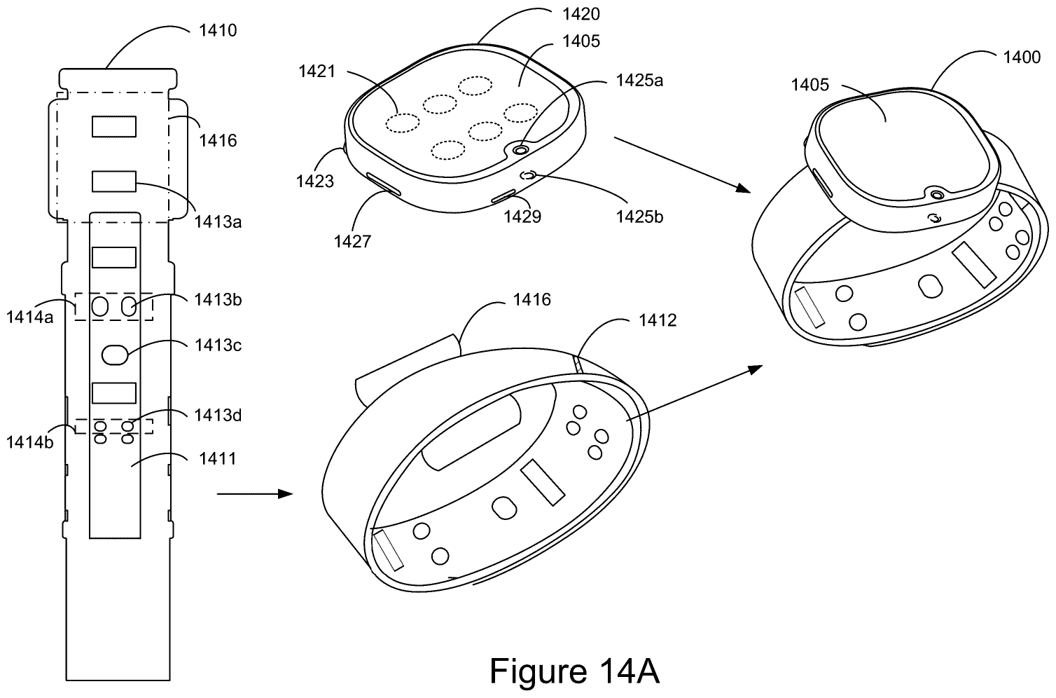

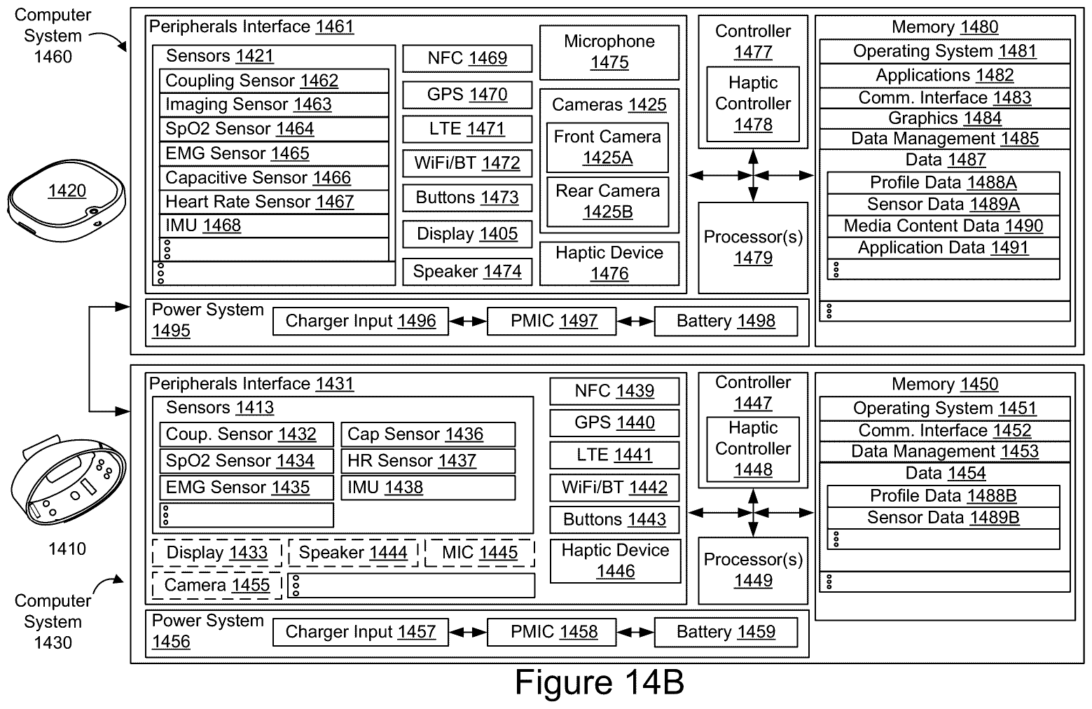

The strain-relief layer 108 is coupled with the FPC 106 and/or the overmolded metal layer 110 through adhesive layers 202. In some embodiments, the strain-relief layer 108 sandwiched by one or more adhesive layers 202. The FPC 106 is configured to communicatively couple with the AFE 204, the receiving structure 112, and other electronics components of the wearable device 100 (described below in reference to FIGS. 14A and 14B). The receiving structure 112 is configured to couple with the biopotential-signal sensor structure 104 such that the biopotential-signal sensor structure 104 is electrically coupled with the FPC 106. Specifically, the receiving structure 112 is configured to couple with the coupling portion 206 of the biopotential-signal sensor structure 104. In some embodiments, the edge support structure 216 is applied to adhere to the receiving structure 112. In some embodiments, molding structures (e.g., the low pressure molding 212 and textile 214) are applied to seal and/or encapsulate the stack-up of components as shown in the cross-sectional view of FIG. 1B. In some embodiments, the textile 214 can be an elastomer overmold.

The overmolded metal layer 110 is configured to support at least the FPC 106 and the strain-relief layer 108. In some embodiments, the overmolded metal layer 106 includes one or more magnetic retention portions and/or tabs. The one or more magnetic retention portions and/or tabs are configured to couple with a clasp or other coupling mechanisms that couple the wearable device 100 with a portion of the user's body. In addition, the band overmold 210 is coupled with the overmolded metal layer 110.

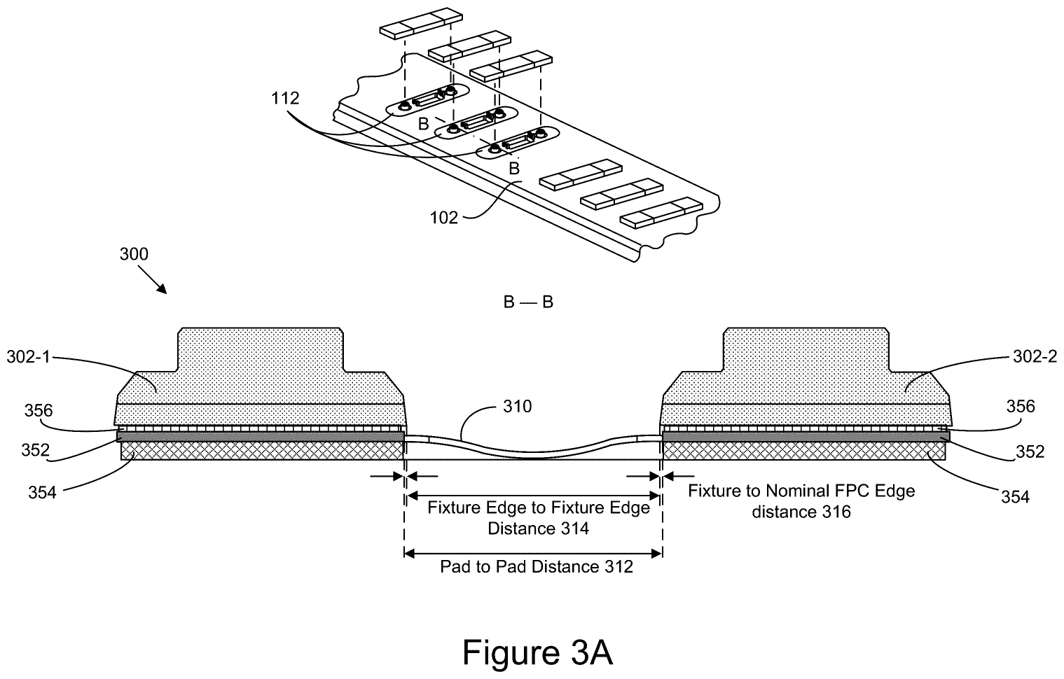

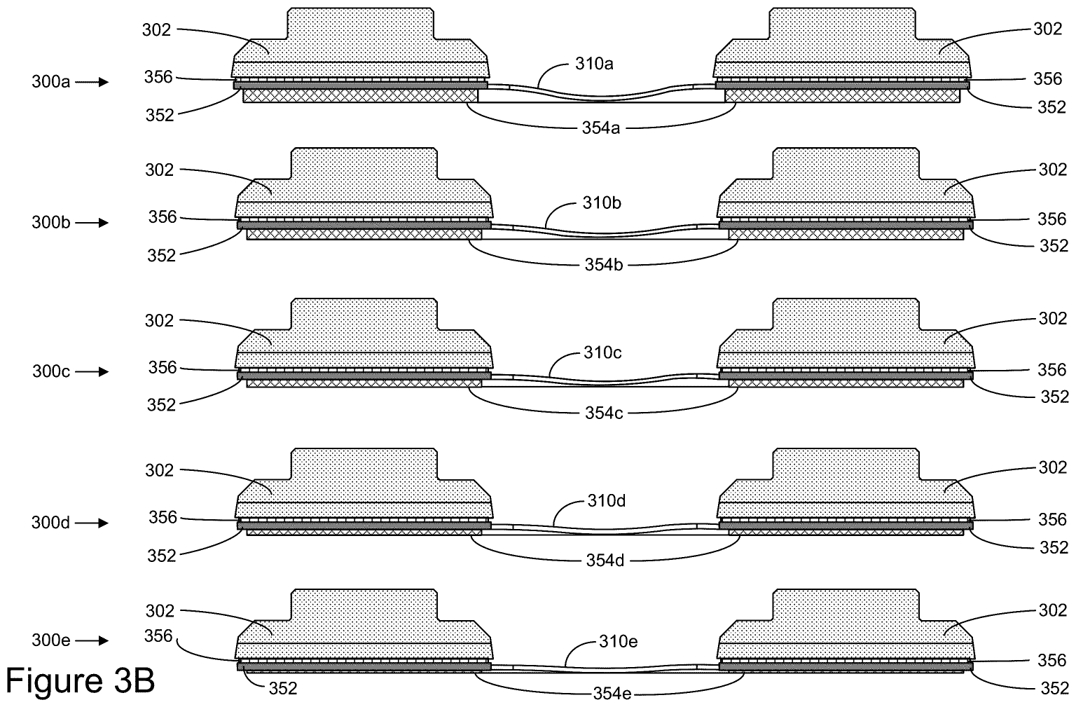

FIGS. 3A-3B illustrate different views along a lengthwise portion of the band portion 102, in accordance with some embodiments. FIG. 3A shows an example cross-sectional view 300 along the lengthwise portion of the band portion 102, and FIG. 3B shows cross-sectional views 300 along the lengthwise portion of the band portion 102 including strain-relief layers with different thicknesses.

FIG. 3A shows an example of the wearable device 100 described above in reference to FIG. 1A. In particular, FIG. 3A illustrates the perspective cross-sectional view 300 of the receiving structures 112 of a cut through B-B along the lengthwise portion of the band portion 102. The perspective cross-sectional view 300 includes strain-relief layers 354, FPC pad layers 352, adhesive layers 356, electrode fixtures 302 (e.g., a first electrode fixture 302-1 and a second electrode fixture 302-2), and a bridging FPC portion 310 of the FPC pad layers 352. Although not shown, in some embodiments, a bridging strain-relief layer is a continuous portion of the strain-relief layers 354 that uniformly connects portions of the strain-relief layers 354. In some embodiments, an FPC layer includes one or more FPC pad layers 352 and one or more FPC bridging portions 310.

As shown in the perspective cross-sectional view 300, the FPC pad layers 352 are coupled with the strain-relief layers 354. The FPC pad layers 352 are further coupled with the electrode fixtures 302 via the adhesive layers 356. The bridging FPC portion 310 has a length (e.g., an interchannel length) greater than the distance between the edges of the first electrode fixture 302-1 and the second electrode fixture 302-2 such that there is extra length between the respective electrode fixtures to reduce strain (e.g., provide additional slack such that the FPC layer is not outstretched). For example, as shown in the perspective cross-sectional view 300, the length of the bridging FPC portion 310 is longer than a pad-to-pad distance 312 and a fixture edge to fixture edge distance 314. The pad-to-pad distance 312 is defined as the distance between the edges of the respective FPC pad layers 352 of two neighboring receiving structures 112 along the lengthwise portion of the band portion 102. The fixture edge to fixture edge distance 314 is defined as the distance between the edges of the electrode fixtures 302 (e.g., the first electrode fixture 302-1 and the second electrode fixture 302-2) of two neighboring receiving structures 112 along the lengthwise portion of the band portion 102. In some embodiments, the pad-to-pad distance 312 can be 5.00 mm and the fixture edge to fixture edge distance 316 can be 4.88 mm, which yields a fixture to nominal FPC edge distance 316 of 0.06 mm. In some embodiments, the fixture to nominal FPC edge distance 316 includes a fixture tolerance of 0.01 mm and a FPC edge tolerance of 0.05 mm. As the skilled artisan will appreciate upon reading the descriptions provided herein, actual values of the pad-to-pad distance 312, the fixture edge to fixture edge distance 314, and the fixture to nominal FPC edge distance 316 can vary, subject to design specifications and manufacturing tolerances in an assembly process.

FIG. 3B shows different combinations of the bridging FPC portion 310 length and the strain-relief layer 354 thickness. Specifically, FIG. 3B shows five distinct embodiments (e.g., first, second, third, fourth, and fifth cross-sectional views of a lengthwise portion of the band portion 300a, 300b, 300c, 300d, and 300e) that include distinct bridging FPC portion 310 (e.g., first, second, third, fourth, and fifth bridging FPC portions 310a, 310b, 310c, 310d, and 310e) lengths and strain-relief layer 354 (e.g., first, second, third, fourth, and fifth strain-relief layers 354a, 354b, 354c, 354d, and 354e) thicknesses. In particular, each combination of the bridging FPC portion 310 length and the strain-relief layer 354 thickness is associated with a respective flat to folded length delta of the bridging FPC portion 310. The flat to folded length delta of the bridging FPC portion 310 is represented as the difference between the corresponding bridging FPC portion 310 length and the corresponding pad-to-pad distance 312.

Table 1 shows a quantitative relationship between the strain-relief layer 354 thickness and the flat to folded length delta of the bridging FPC portion 310. As shown in Table 1 (reproduce below), the flat to folded length delta of the bridging FPC portion 310 decreases with a decrease in the strain-relief layer 354 thickness (e.g., the thinner the strain-relief layer 354 the less the bridging FPC portion 310 length can change). Potential advantages of using a thinner strain-relief layer include less overall band thickness while maintaining durability of the wearable device and reduced manufacturing costs resulting from less material usage.

TABLE 1

| | Strain-relief layer | Flat to Folded |
|---|---|---|
| Embodiments | Thickness (mm) | Length Delta (mm) |
| 300a | 0.25 | 0.0483 |
| 300b | 0.20 | 0.0357 |
| 300c | 0.15 | 0.0245 |
| 300d | 0.10 | 0.0148 |
| 300e | 0.05 | 0.0069 |

As shown in Table 1, the flat to folded length deltas of the bridging FPC portion 310 are based on the thickness of the strain-relief layer 354 such that thinner strain-relief layers 354 result in lower flat to folded length deltas. For example, in the first cross-sectional view of a lengthwise portion of the band portion 300a, the flat to folded length delta of the first bridging FPC portion 310a is 0.0483 mm, while the flat to folded length delta is 0.0357 mm for the second cross-sectional view of a lengthwise portion of the band portion 300b. In other words, a change of 0.05 mm in the strain-relief layer thickness results in a corresponding change of 0.0126 mm in the flat to folded length deltas. The change in the flat to folded length deltas due to strain-relief layer 354 can be used to design the band portion 102 of the wearable device 100 based on the expected and/or predicted use cases of the wearable device. Specifically, the flat to folded length deltas of the bridging FPC portions 310 can be used to design wearable devices that position electrode fixtures 302 at predetermined locations for a particular use case such that the electrode fixtures 302 of the wearable devices contact the user's skin and capture reliable data. In some embodiments, the different strain-relief layer 354 thicknesses minimally alter the pad-to-pad distance 312 when the wearable device is taught or pulled. Additionally, the strain-relief layers are resistant to stretching (e.g., minimum stretching experienced regardless of thickness).

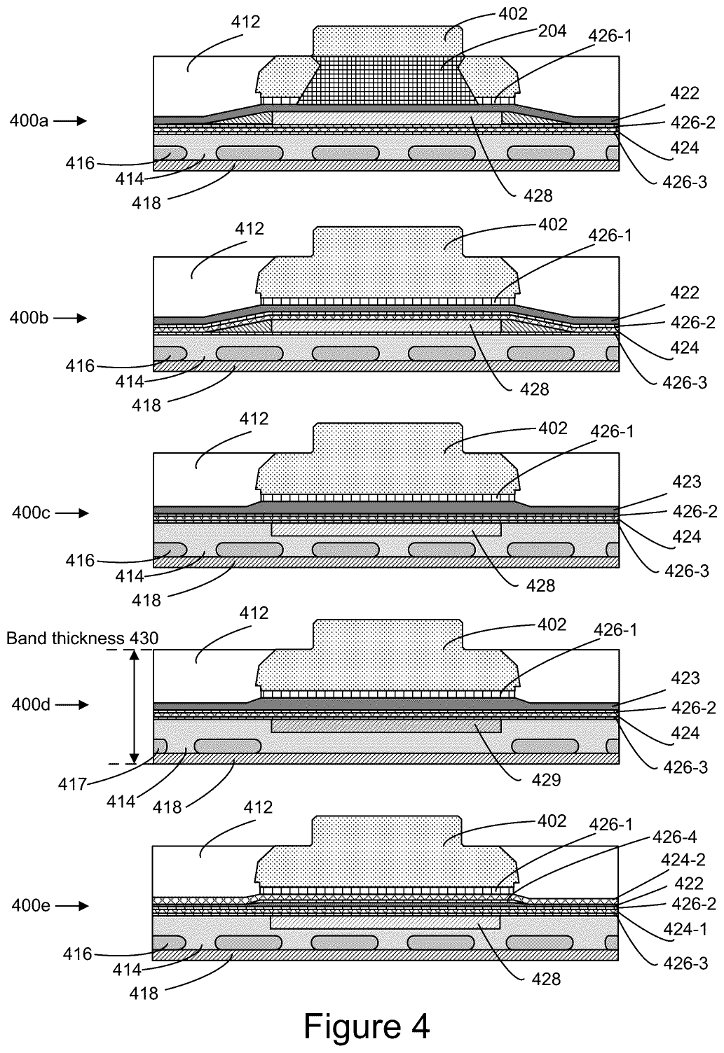

FIG. 4 illustrates cross-sectional views of example receiving structures, in accordance with some embodiments. The receiving structure cross-sectional views show different stack-up embodiments (e.g., first, second, third, fourth, and fifth stack-up embodiments 400a, 400b, 400c, 400d, and 400e). The different stack-up embodiments can be used with the wearable device 100 described above in reference to FIGS. 1A-1C. Each stack-up embodiment includes at least a textile 418 (e.g., or elastomer overmold), a clasp retention mechanism 416 (e.g., an overmolded metal layer 110 as shown in FIGS. 1A and 2), a band overmold 414, a lower pressure molding 412, one or more strain-relief layers 424, one or more adhesive layers 426, an FPC layer 422, an electrode fixture 402, and a stiffener 428. The molding structures (e.g., the low pressure molding 412, the band overmold 414, and the textile 418) are applied to seal and/or encapsulate one or more components of the stack-up embodiments (e.g., the FPC layer 422, the clasp retention mechanism 416, etc.). In some embodiments, the clasp retention mechanism 416 includes one or more magnetic retention portions and/or tabs.

Turning to the first stack-up embodiment 400a, the strain-relief layer 424 is disposed between at least two adhesive layers 426-2 and 426-3. The strain-relief layer 424 and the FPC layer 422 are configured to couple with the stiffener 428. In particular, the FPC layer 422 is bent to accommodate the stiffener 428. The electrode fixture 402 couples with the FPC layer 422 through the adhesive layer 426-1. The first stack-up embodiment 400a further includes an AFE 204 attached to the FPC layer 422.

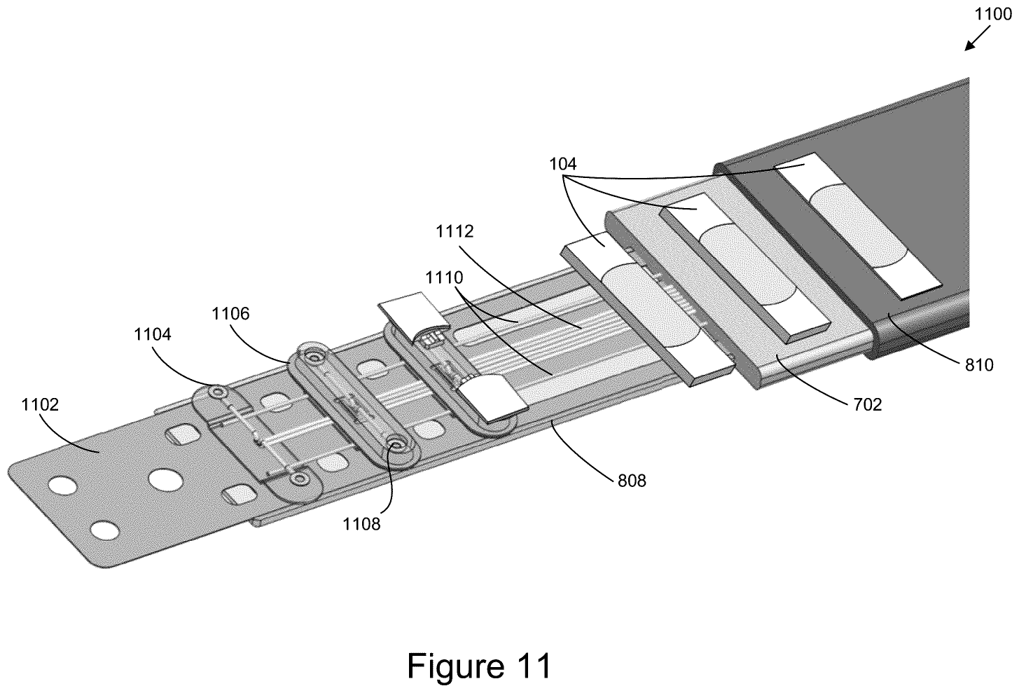

In the second stack-up embodiment 400b, the stiffener 428 is positioned under the strain-relief layer 424 and coupled with the band overmold 414 through the adhesive layer 426-3. To accommodate this change, the strain-relief layer 424 is configured to couple with the FPC layer 422 via the adhesive layer 426-2, and a combined stack of the strain-relief layer 424, the adhesive layer 426-2, and the FPC layer 422 is bent to accommodate the stiffener 428. Similar to the first embodiment 400a, the electrode fixture 402 couples with the FPC layer 422 through the adhesive layer 426-1. The second stack-up embodiment 400b offloads the AFE 204 (e.g., to a computer system of communicatively coupled with a wearable device, such as computer system 1460; FIGS. 14A and 14B), which allows for mechanical structure optimization as discussed below. By offloading the AFE 204, the second stack-up embodiment 400b allows for the stiffener 428 width and/or receiving structure width to be reduced and improves compatibility with flexible electrodes and/or receivers. In addition, the second stack-up embodiment 400b improves reliability, such as the capability to place the FPC layer 422 on a neutral axis, and mitigates reliability risk associated with surface-mount technology (SMT) components (e.g., AFEs 204). Additional detail on offloading the AFEs is provided below in reference to FIG. 11. The second stack-up embodiment 400b is interchangeable with the first stack-up embodiment 400a (e.g., the first and/or the second stack-up embodiments 400a and 400b can be used in the same wearable device architecture).

In the third stack-up embodiment 400c, the stiffener 428 is disposed within the band overmold 414 (e.g., under the distinct FPC layer 423 and the strain-relief layer 424). This allows the distinct FPC layer 423 and the strain-relief layer 424 to be flattened or disposed substantially planarly (e.g., without bending to accommodate one or more components of the third stack-up embodiment 400c). In some embodiments, the strain-relief layer 424 of the third stack-up embodiment 400-3 is optional. Similar to the first stack-up embodiment 400a, the strain-relief layer 424 of the third stack-up embodiment 400c is sandwiched by two adhesive layers 426-2 and 426-3, and the electrode fixture 402 couples with the the FPC layer 423 through the adhesive layer 426-1. Additionally, similar to the second stack-up embodiment 400b, the third stack-up embodiment 400c offloads the AFE 204 (e.g., to a computer system communicatively coupled with the wearable device). The third stack-up embodiment 400c provides the same benefits to those described above with respect to the second stack-up embodiment 400b. The third stack-up embodiment 400c is not interchangeable with the first and second stack-up embodiments 400a and 400b.

In the fourth stack-up embodiment 400d, the thickness of the band portion 102 (e.g., band thickness 430) is decreased. To reduce the overall the band thickness 430, the fourth stack-up embodiment 400d uses a distinct stiffener 429 and/or a distinct clasp retention mechanism 417. Similar to the third stack-up embodiment 400c, the distinct stiffener 429 is disposed within the band overmold 414 (e.g., under the distinct FPC layer 423 and the strain-relief layer 424). Different from the third stack-up embodiment 400c, the distinct clasp retention mechanism 417 is not disposed underneath the distinct stiffener 429 (e.g., reducing the overall thickness of the fourth stack-up embodiment 400d). Additionally, the distinct stiffener 429 is molded into the band overmold 414 and/or includes magnetic elements (e.g., which allows a clasp to couple with the distinct stiffener 429 if positioned at that location). The fourth stack-up embodiment 400d can use the distinct FPC layer 423 described above in reference to the third stack-up embodiment 400c. In some embodiments, optimization of the band thickness 430 can be achieved by varying the thickness of each layer shown in the fourth stack-up embodiment 400d. In some embodiments, optimization of the band thickness 430 adheres design specifications and reliability requirements. Additionally, in some embodiments, the strain-relief layer 424 in the fourth stack-up embodiment 400d is optional (e.g., which decreases a thickness, width, height, and/or other size of the wearable device). The fourth stack-up embodiment 400d provides the same benefits to those described above with respect to the second and third stack-up embodiments 400b and 400c. The fourth stack-up embodiment 400b is not interchangeable with the first and second stack-up embodiments 400b and 400c.

The fifth stack-up embodiment 400e includes one or more features described above in reference to the first through fourth stack-up embodiments 400a through 400e. The fifth stack-up embodiment 400e includes a strain-relief sleeve (e.g., formed by a first and a second strain-relief layers 424-1 and 424-2). The strain-relief sleeve is configured to wrap or encase the FPC layer 422, which is coupled to the first and second strain-relief layers 424-1 and 424-2 via the second and a fourth adhesive layers 426-2 and 426-4. The strain-relief sleeve is further configured to couple with the electrode fixture 402 and the stiffener 428 via respective adhesive layers (e.g., the first and third adhesive layers 426-1 and 426-3, respectively). The strain-relief sleeve is configured to improve reliability and mechanical robustness, as well as durability of the wearable device. Moreover, the fifth stack-up embodiment 400e provides the same benefits to those described above with respect to the second through fourth stack-up embodiments 400b through 400d.

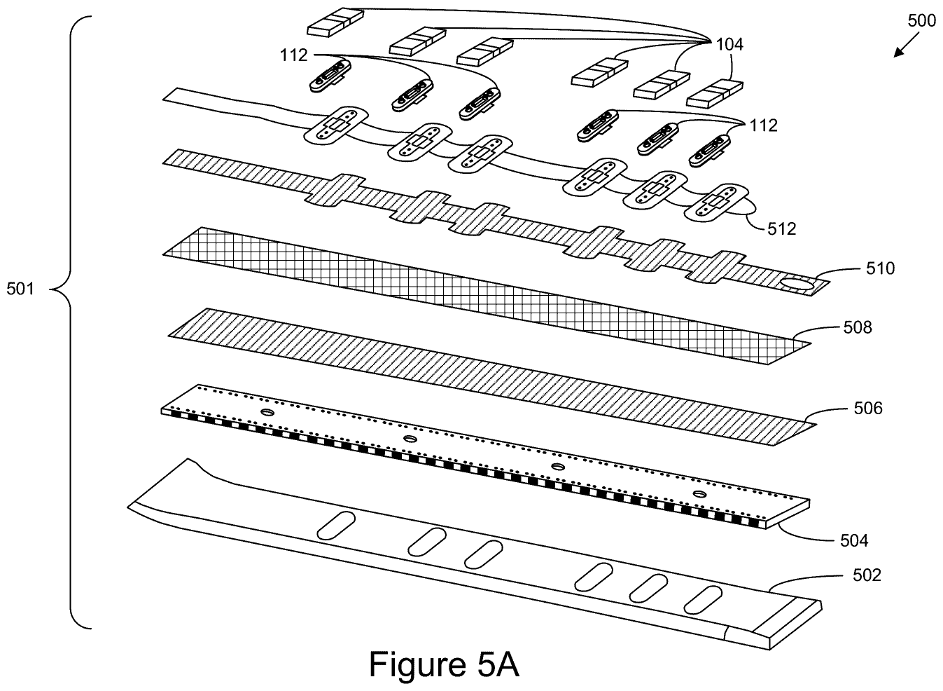

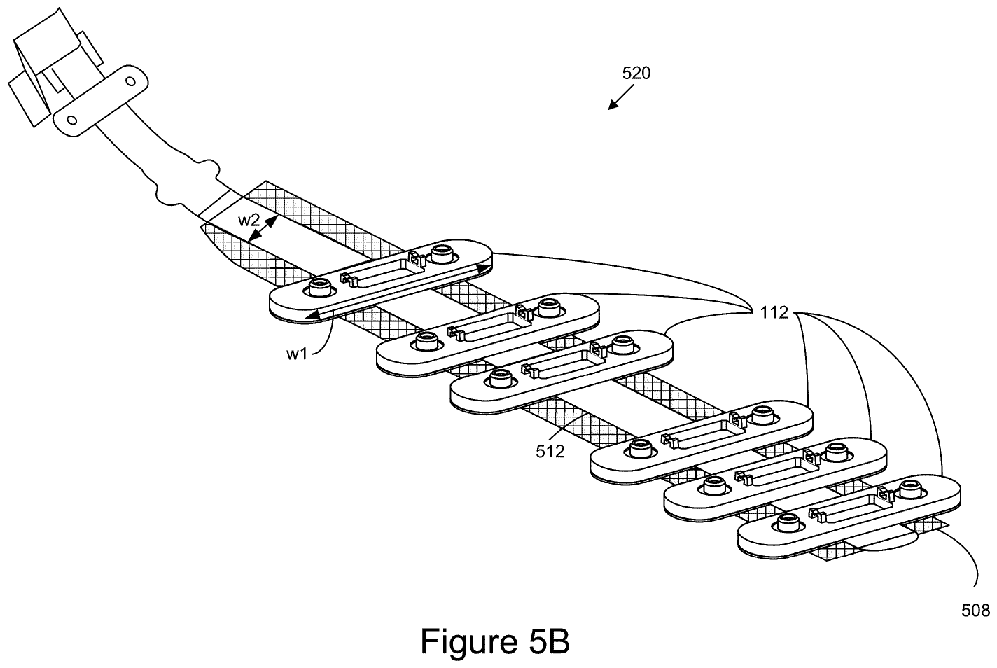

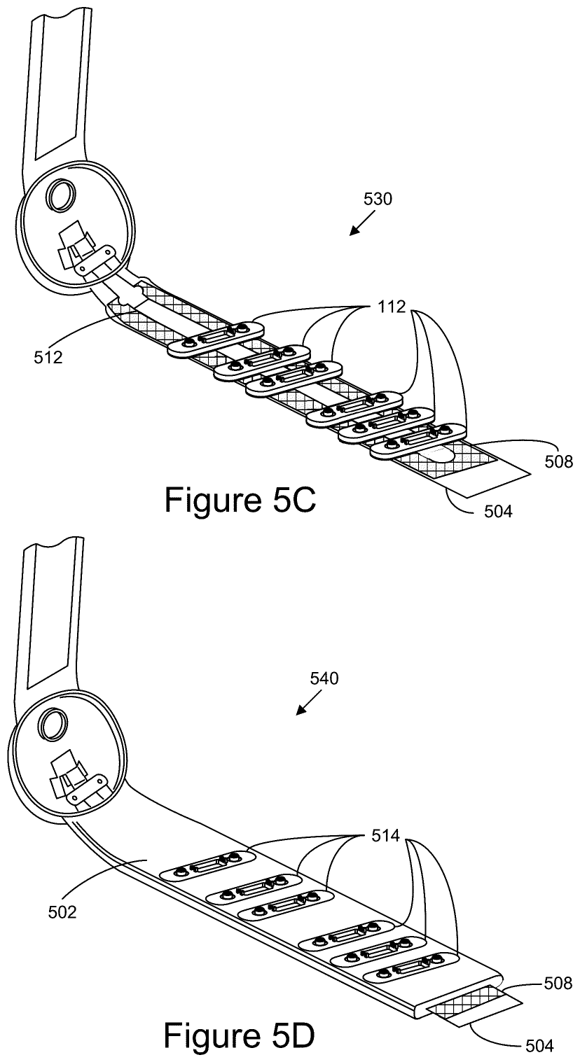

FIGS. 5A-5D illustrate a first example embodiment of the band portion 501 of the wearable device 100, in accordance with some embodiments. The first example embodiment of the band portion 501 and/or components thereof are instances of the band portion 102 and its respective components described above in reference to FIGS. 1A-4. The first example embodiment of the band portion 501 includes, at least, an elastomer overmold 502, an overmolded metal layer 504, a first adhesive layer 506, a strain-relief layer 508, a second adhesive layer 510, an FPC layer 512, one or more receiving structures 112, and one or more biopotential-signal sensing structures 104 (which couple with the receiving structures 112). In some embodiments, each receiving structure 112 houses one or more AFEs and/or electrode fixtures (e.g., exposed portions that are configured to affix the biopotential-signal sensing structures 104 as described above in reference to FIG. 1C). Alternatively, in some embodiments, the one or more AFEs are offloaded to a computing system (e.g., a computer system of communicatively coupled with a wearable device, such as computer system 1460; FIGS. 14A and 14B). In some embodiments, the first example embodiment of the band portion 501 uses a textile in place of the elastomer overmold 502.

FIG. 5A shows an exploded view 500 of the first example embodiment of the band portion 501 of the wearable device 100. Specifically, FIG. 5A shows an example alignment and orientation of one or more layers of the band portion 501, in accordance with some embodiments. In the first example embodiment of the band portion 501, the strain-relief layer 508 is substantially uniform (e.g., a substantially rectangular shape) and extends to the length of the FPC layer 512 and/or the overmolded metal layer 504. In some embodiments, the strain-relief layer 508 is configured to couple with the FPC layer 512 and/or the overmolded metal layer 504 via one or more adhesive layers, such as the second adhesive layers 510 and the first adhesive layer 506, respectively.

The FPC layer 512 extends along a lengthwise portion of the band portion 102. The FPC layer 512 includes one or more portions (e.g., substantially oval portions) that define outlines for the one or more receiving structures 112 and are configured to couple with one or more AFEs and/or electrode fixtures. While the one or more portions of the FPC layer 512 are substantially oval, the one or more portions can be any other shape (e.g., circular, rectangular, etc.) or based on a shape of the one or more receiving structures 112. As described above, the FPC layer 512 couples with the strain-relief layer 508 via the second adhesive layer 510. In addition, the FPC layer 512 and the second adhesive layer 510 have a substantially similar shape and length.

The one or more receiving structures 112 are configured to couple with the one or more portions (e.g., substantially oval portions) of the FPC layer 512. The one or more receiving structures 112 are also configured to couple with the one or more biopotential-signal sensing structures 104.

The overmolded metal layer 504 extends along a lengthwise portion of the band portion 102. The overmolded metal layer 504, similar to the strain-relief layer 508, is substantially uniform. For example, the overmolded metal layer 504 is substantially rectangular. As described above, the overmolded metal layer 504 couples with the strain-relief layer 508 via the first adhesive layer 506. The overmolded metal layer 504 and the first adhesive layer 506 have a substantially similar shape. In some embodiments, the overmolded metal layer 504 includes one or more magnetic retentions or paramagnetic tabs for coupling with a clasp mechanism (not shown) of the band portion 102. In this way, the overmolded metal layer 504 and the clasp mechanism allow a user to adjust a size of the wearable device 100.

The elastomer overmold 502 (or textile) is disposed over at least the overmolded metal layer 504, the first adhesive layer 506, the strain-relief layer 508, the second adhesive layer 510, and the FPC layer 512. The elastomer overmold 502 is disposed around the one or more receiving structures 112 to allow for the one or more biopotential-signal sensing structures 104 to be coupled with the one or more receiving structures 112. The elastomer overmold 502 is configured to protect the internal components of the band portion 102 as well as provide a surface that can interface with skin of the user. In some embodiments, the elastomer overmold 502 is a cosmetic elastomer or rubber band and interchangeable with a textile or used in conjunction with a textile.

FIG. 5B shows a partial stack-up embodiment 520 of the band portion 501. The partial stack-up architecture 520 of the band portion 501 includes at least the strain-relief layer 508, the FPC layer 512 (e.g, coupled with the strain-relief layer 508 via the second adhesive layer 510 as described above in reference to FIG. 5A), and the one or more receiving structures 112. As mentioned above, the strain-relief layer 508 is substantially uniform (e.g., a substantially rectangular shape) and extends to the length of the FPC layer 512, while the FPC layer 512 includes one or more portions for coupling with one or more receiving structures 112. As shown in FIG. 5B, the FPC layer 512 has non-uniform widths—the one or more (oval) portions having a first width (w1) and the remaining FPC layer 512 having a second width (w2). The width of the strain-relief layer 508 is less than the first width w1 of the FPC layer 512 but greater than the second width w2 of the FPC layer 512. In some embodiments, the dimensions of the strain-relief layer 508 can vary, subject to the reliability requirements and other design specifications. In some embodiments, enhancing robustness and reliability may necessitate customization of the strain-relief layer 508, which can increase design complexity. For example, as shown in FIG. 3B, a requirement of less overall band thickness necessities a use of a thinner strain-relief layer while maintaining durability of the wearable device.

FIG. 5C shows an additional partial stack-up architecture 530 of the band portion 501. The additional partial stack-up architecture 530 shows the partial stack-up embodiment 520 of the band portion 501 (e.g., at least the strain-relief layer 508, the FPC layer 512, and the receiving structures 112) further coupled with the overmolded metal layer 504 (e.g., via the first adhesive layer 506 (FIG. 5A)). The overmolded metal layer 504, the strain-relief layer 508, and the first adhesive layer 506 have a substantially similar shape (e.g., a substantially uniform shape). For example, the overmolded metal layer 504 is substantially rectangular. The overmolded mental layer 506 has a substantially similar width to that of the strain-relief layer 508 and can extend beyond the length of the strain-relief layer 508. In some embodiments, the dimensions of the overmolded layer 504 can vary, subject to the reliability requirements and other design specifications.

FIG. 5D shows an elastomer overmold 502 disposed over the additional partial stack-up architecture 530 (forming an enclosed stack-up architecture 540 of the band portion 501). The elastomer overmold 502 encapsulates at least the overmolded metal layer 504, the strain-relief layer 508, the FPC layer 512, and a portion of the one or more receiving structures 112. As mentioned above, the elastomer overmold 502 is disposed around the one or more receiving structures 112 to allow for the one or more biopotential-signal sensing structures 104 (FIG. 5A) to be coupled with the one or more receiving structures 112. The elastomer overmold 502 is configured to protect the internal components of the band portion 501 as well as provide a surface that can interface with skin of the user.

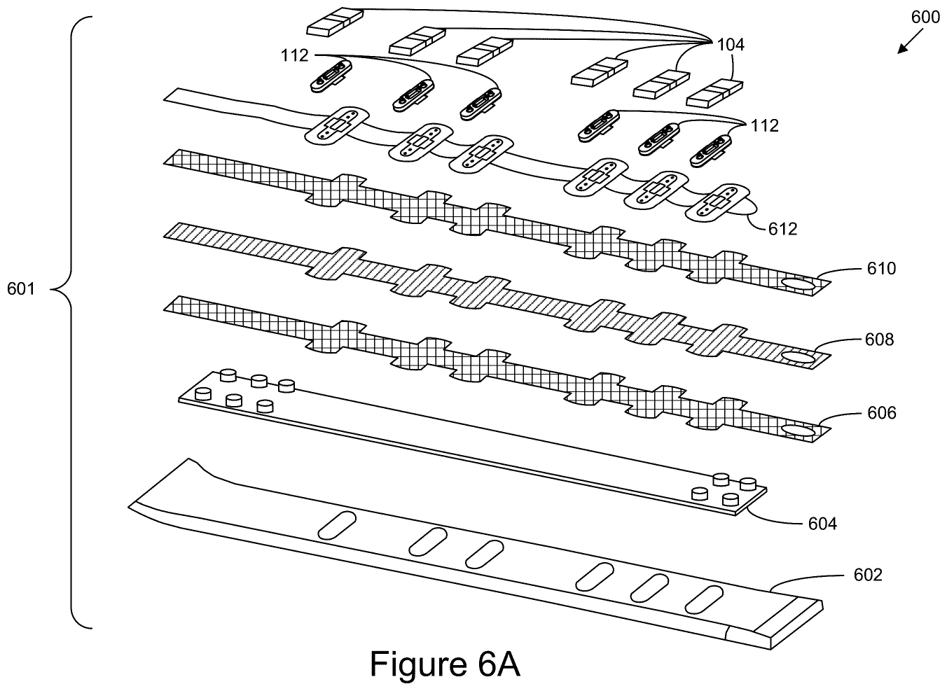

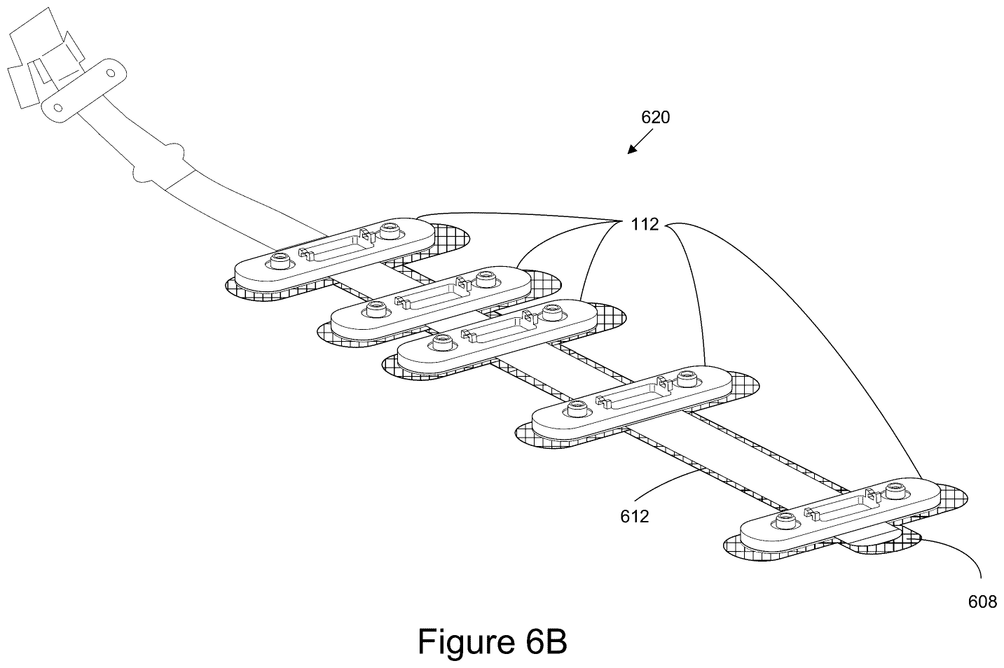

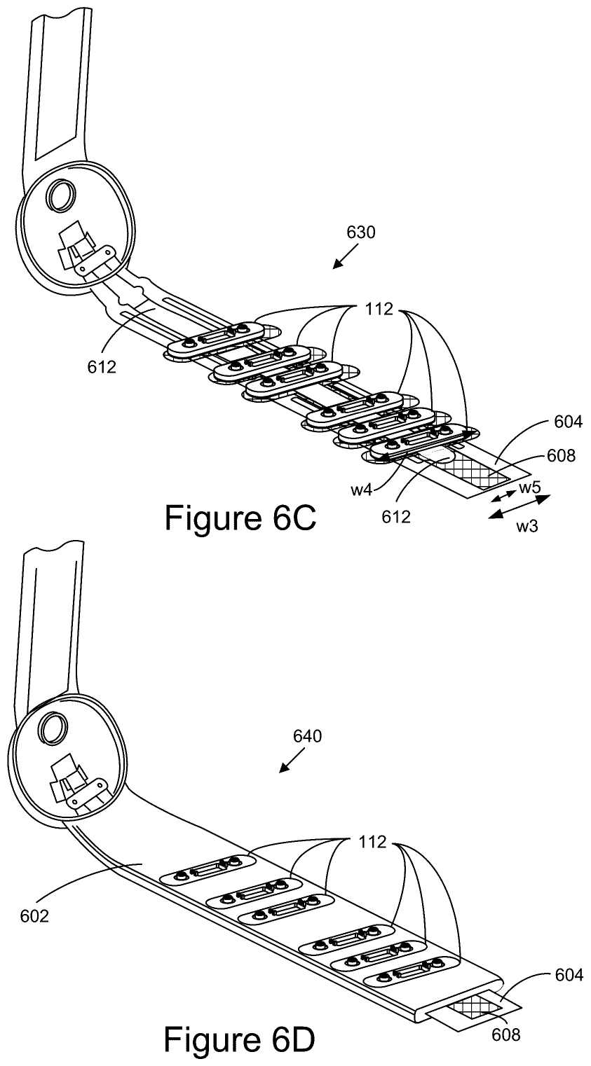

FIGS. 6A-6D illustrate a second example embodiment of the band portion 601 of the wearable device 100, in accordance with some embodiments. The second example embodiment of the band portion 501 and/or components thereof are instances of the band portion 102 and its respective components described above in reference to FIGS. 1A-4. Similar to the first example embodiment of the band portion 501, the second example embodiment of the band portion 601 includes, at least, an elastomer overmold 602, an overmolded metal layer 604, a first adhesive layer 606, a strain-relief layer 608, a second adhesive layer 610, an FPC layer 512, one or more receiving structures 112, and one or more biopotential-signal sensing structures 104 (which couple with the receiving structures 112). Different from the strain-relief layer 508 of the first example embodiment of the band portion 501, the strain-relief layer 608 of the second example embodiment of the band portion 601 is not uniform. Similar to the first example embodiment of the band portion 501, in some embodiments, AFEs are housed within receiving structures 112 or offloaded to a computing system (e.g., a computer system of communicatively coupled with a wearable device, such as computer system 1460; FIGS. 14A and 14B). In some embodiments, the second example embodiment of the band portion 601 uses a textile in place of the elastomer overmold 602.

FIG. 6A shows an exploded view 600 of the second example embodiment of the band portion 601 of the wearable device 100. Specifically, FIG. 6A shows an example alignment and orientation of one or more layers of the band portion 601, in accordance with some embodiments. Similar to the first example embodiment of the band portion 501, the FPC layer 612 of the second example embodiment of the band portion 601 extends along a lengthwise portion of the band portion 102. The FPC layer 612 includes one or more portions (e.g., substantially oval portions) that define outlines for coupling with the one or more receiving structures 112 and are configured to couple with one or more AFEs and/or electrode fixtures. As described above in reference to FIGS. 5A-5D, the one or more portions of the FPC layer 612 can include a shape based on a shape of the one or more receiving structures 112. As described above, the FPC layer 612 couples with the strain-relief layer 608 via the second adhesive layer 610. Similarly, the FPC layer 612 and the second adhesive layer 610 have a substantially similar shape and length.

Accordingly, different from the strain-relief layer 508 of the first example embodiment of the band portion 501, the strain-relief layer 608 is not uniform and includes one or more portions (e.g., substantially oval portions) that define outlines for the FPC layer 612. Similarly, the strain-relief layer 608 extends to the length of the FPC layer 612 and/or the overmolded metal layer 604. In other words, the strain-relief layer 608 substantially copies or duplicates the outline and/or shape of the FPC layer 612. In some embodiments, the strain-relief layer 608 is configured to couple with the FPC layer 612 and/or the overmolded metal layer 604 via one or more adhesive layers, such as the second adhesive layers 610 and the first adhesive layer 606, respectively.

The overmolded metal layer 604 extends along a lengthwise portion of the band portion 102. As described above, the overmolded metal layer 604 couples with the strain-relief layer 608 via the first adhesive layer 606. Different from the first example embodiment of the band portion 501, the overmolded metal layer 604 has a substantially uniform shape, while the first adhesive layer 606 has a substantially similar shape to the strain-relief layer 608 (e.g., the first adhesive layer 606 follows outlines for the strain-relief layer 608 and/or the FPC layer 612). In some embodiments, the overmolded metal layer 604 includes one or more magnetic retentions or paramagnetic tabs for coupling with a clasp mechanism (not shown) of the band portion 102. In this way, the overmolded metal layer 604 and the clasp mechanism allow a user to adjust a size of the wearable device 100.

Similar to the first example embodiment of the band portion 501, the elastomer overmold 602 (or textile) is disposed over at least the overmolded metal layer 604, the first adhesive layer 606, the strain-relief layer 608, the second adhesive layer 610, and the FPC layer 612. The elastomer overmold 602 is disposed around the one or more receiving structures 112 to allow for the one or more biopotential-signal sensing structures 104 to be coupled with the one or more receiving structures 112. The elastomer overmold 602 is configured to protect the internal components of the band portion 102 as well as provide a surface that can interface with skin of the user. In some embodiments, the elastomer overmold 602 is a cosmetic elastomer or rubber band and interchangeable with a textile or used in conjunction with a textile.

FIG. 6B shows a partial stack-up embodiment 620 of the band portion 601. Similar to the stack-up architecture 520 shown in FIG. 5B, the partial stack-up architecture 620 of the band portion 601 includes at least the strain-relief layer 608, the FPC layer 612 (e.g, coupled with the strain-relief layer 508 via the second adhesive layer 610 as described above in reference to FIG. 6A), and the one or more receiving structures 112. The strain-relief layer 608 includes one or more portions (e.g., substantially oval portions) that copy or are substantially similar to an outline of the FPC layer 612. As described the above, the one or more portions of the FPC layer 612 outline a shape of and are configured to couple with one or more receiving structures 112. The FPC layer 612 can have dimensions similar to the outline of the FPC layer 512 described above in reference to FIG. 5B (e.g., the FPC layer 612 and the strain-relief layer 608 can have respective varying widths based on a location and dimensions of a receiving structure 112).

FIG. 6C shows an additional partial stack-up architecture 630 of the band portion 601. The additional partial stack-up architecture 630 shows the partial stack-up embodiment 620 of the band portion 601 (e.g., at least the strain-relief layer 608, the FPC layer 612, and the receiving structures 112) further coupled with the overmolded metal layer 604 (e.g., via the first adhesive layer 606 (FIG. 6A)). The overmolded metal layer 604 is substantially uniform, while the strain-relief layer 608 and the first adhesive layer 506 have an outline substantially similar to the FPC layer 612. For example, the overmolded mental layer 604 has a width (w3) that is greater than a first width (w5) of the strain-relief layer 608 and less than a second width (w4) of the strain-relief layer 608. As described above, first width (w5) and the second width (w4) of the of the strain-relief layer 608 depend on the locations and dimensions of the receiving structures 112. In some embodiments, the overmolded metal layer 604 can extend beyond the length of the strain-relief layer 608. In some embodiments, the dimensions of the overmolded layer 604 can vary, subject to the reliability requirements and other design specifications.

FIG. 6D shows an elastomer overmold 602 disposed over the additional partial stack-up architecture 630 (forming an enclosed stack-up architecture 640 of the band portion 601 that protects the internal components of the band portion 601 and provides a surface that can interface with skin of the user), similar to the architecture shown in FIG. 5D. The elastomer overmold 602 encapsulates at least the overmolded metal layer 604, the strain-relief layer 608, the FPC layer 612, and a portion of the one or more receiving structures 112. As mentioned above, the elastomer overmold 602 is disposed around the one or more receiving structures 112 to allow for the one or more biopotential-signal sensing structures 104 (FIG. 6A) to be coupled with the one or more receiving structures 112.

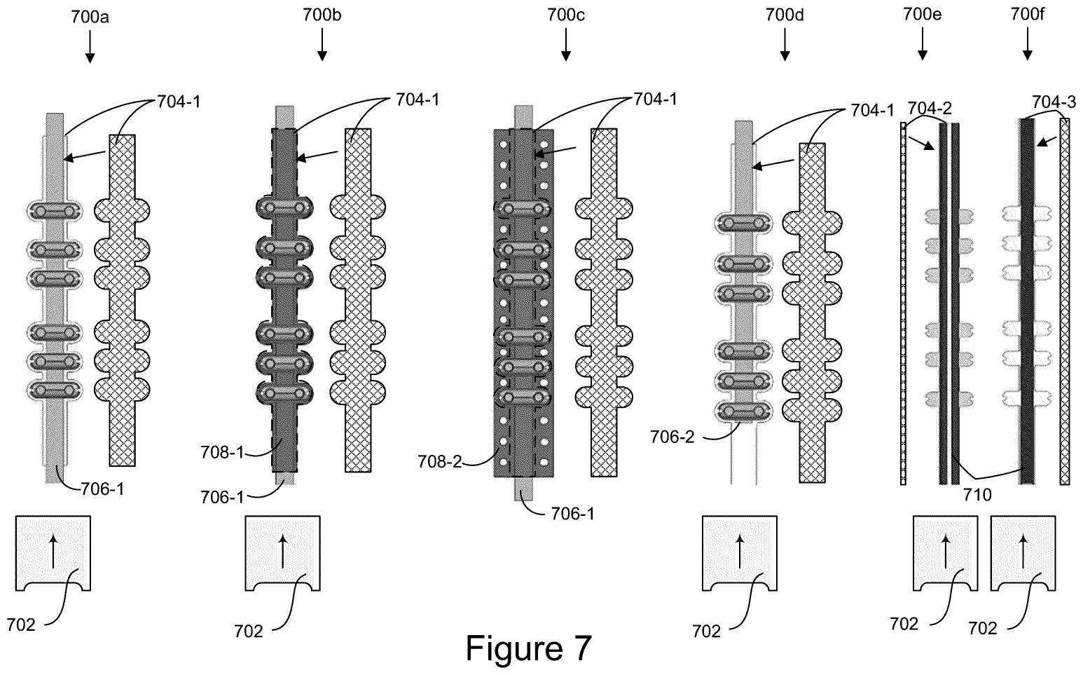

FIG. 7 illustrates different flex stack-up embodiments, in accordance with some embodiments. The different flex stack-up embodiments can be used with any wearable device and/or other components described above in reference to FIGS. 1A-6D. FIG. 7 shows respective top views of first through sixth flex stack-up embodiments (e.g., 700a through 700f). Each flex stack-up embodiment includes at least an FPC layer 706 and at least one other layer for improving the reliability of the FPC layer 706.

The first flex stack-up embodiment 700a includes at least a strain-relief layer 704-1 and a FPC layer 706-1, which are coupled via one or more adhesive layers as discussed above in reference to FIGS. 1A-6D. The first flex stack-up embodiment 700a is further coupled with a band overmold 702 (which can be a textile or elastomer overmold as described above in reference to FIGS. 1A-6D). The FPC layer 706-1 can extend beyond the length of the strain-relief layer 704-1. In some embodiments, the dimensions of the strain-relief layer 704-1 and FPC layer 706-1 can vary, subject to the reliability requirements and other design specifications.

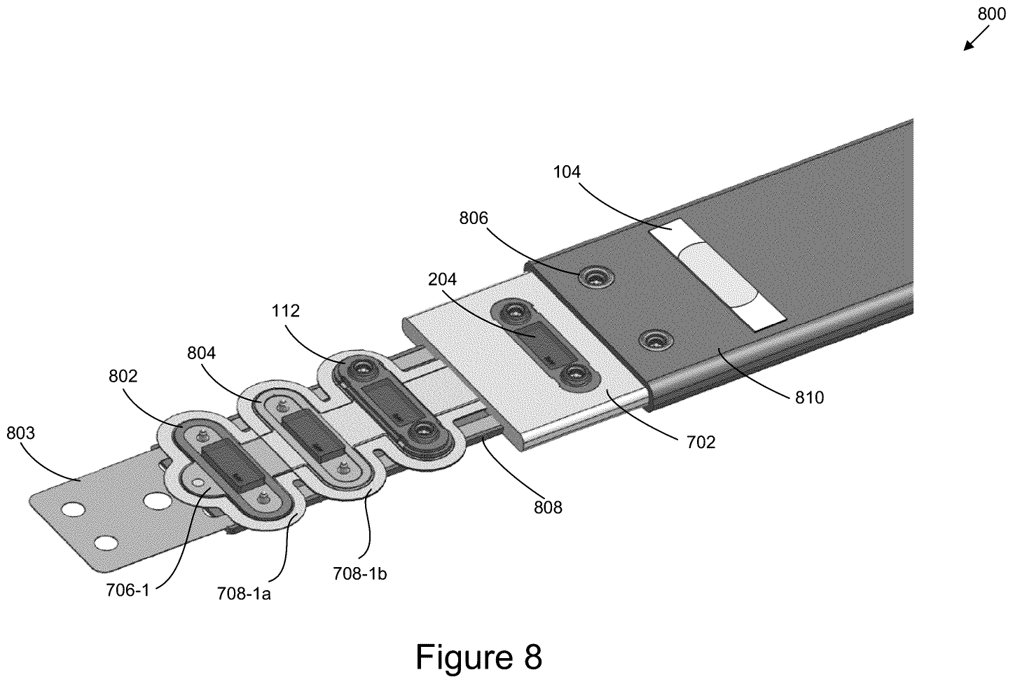

The second flex stack-up embodiment 700b includes at least the FPC layer 706-1 and a wrapper 708-1. The one or more layers of the second flex stack-up embodiment 700b can be coupled with one or more adhesive layers as discussed above. The wrapper 708-1 can be used in place of the strain-relief layer 704-1. Alternatively, in some embodiments, the second flex stack-up embodiment 700b includes a strain-relief layer 704-1 and the wrapper 708-1. As shown by the second flex stack-up embodiment 700b, the FPC layer 706-1 is encapsulated by the wrapper 708-1. The wrapper 708-1 and the FPC layer 706-1 have a substantially similar shape (e.g., rectangular portions and oval portions). In some embodiments, the wrapper 708-1 dimensions are larger than the dimension of the FPC layer 706-1 so that the FPC layer 706-1 is fully covered by the wrapper 708-1. The second flex stack-up embodiment 700b can further includes the band overmold 702 to enclose at least the wrapper 708-1 and the FPC layer 706-1. The second flex stack-up embodiment 700b is configured to reduce the stress on the FPC layer 706-1. Additional details on the second flex stack-up embodiment 700b is provided below in reference to FIG. 8. In some embodiments, the wrapper 708b is a polymer or other material for sealing a channel.

The third flex stack-up embodiment 700c includes at least the FPC layer 706-1 and a uniform wrapper 708-2. The uniform wrapper 708-2 is a substantially uniform shape (e.g., a substantially rectangular shape). The uniform wrapper 708-2 is larger than the wrapper 708-1. The uniform wrapper 708-2 establishes a rigid frame by encapsulating the FPC layer 706-1. The uniform wrapper 708-2 can be used in place of the strain-relief layer 704-1. Alternatively, in some embodiments, the third flex stack-up embodiment 700c also includes a strain-relief layer 704-1. The one or more layers of the third flex stack-up embodiment 700c can be coupled via one or more adhesive layers as discussed above. In the third flex stack-up embodiment 700c, the band overmold 702 is optional. The third flex stack-up embodiment 700c can be used to reduce the thickness of a band portion of a wearable device. The wrapper 708-1 and the uniform wrapper 708-2 are formed of similar materials.

The fourth flex stack-up embodiment 700d includes at least the strain-relief layer 704-1 and a decoupled FPC layer 706-2, which are coupled via one or more adhesive layers. The decoupled FPC layer 706-2 is decoupled on one side of the strain-relief layer 704-1. In some embodiments, the decoupled FPC layer 706-2 is coupled to a computer system via a single end (e.g., computer system 1460; FIGS. 14A and 14B). By decoupling a portion of the FPC layer 706-2 less strain is transferred to the decoupled FPC layer 706-2. The fourth flex stack-up embodiment 700d includes the band overmold 702. In some embodiments, the dimensions of the strain-relief layer 704-1 and the decoupled FPC layer 706-2 can vary, subject to the reliability requirements and other design specifications.

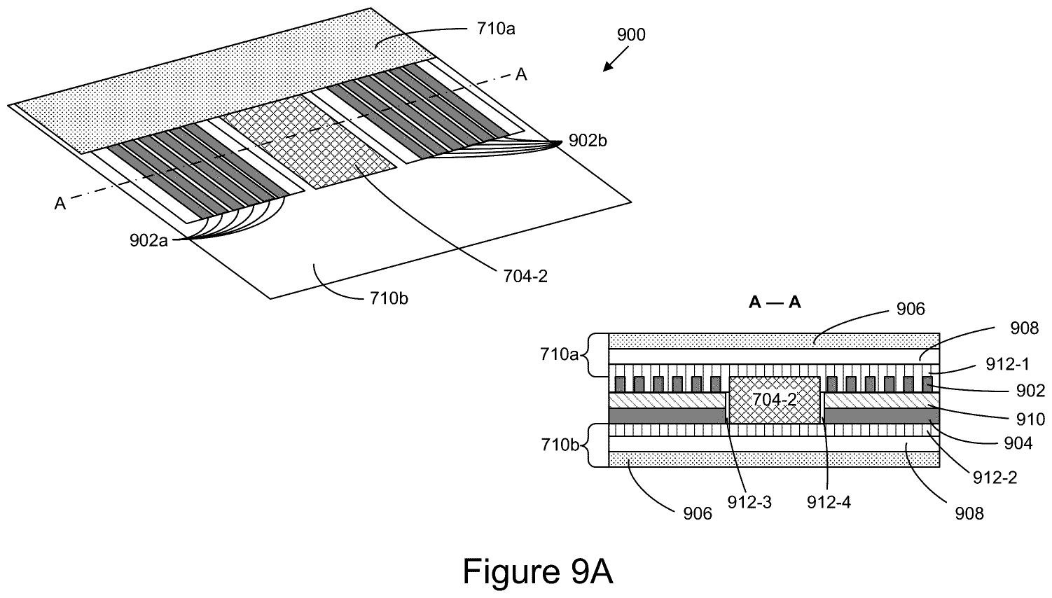

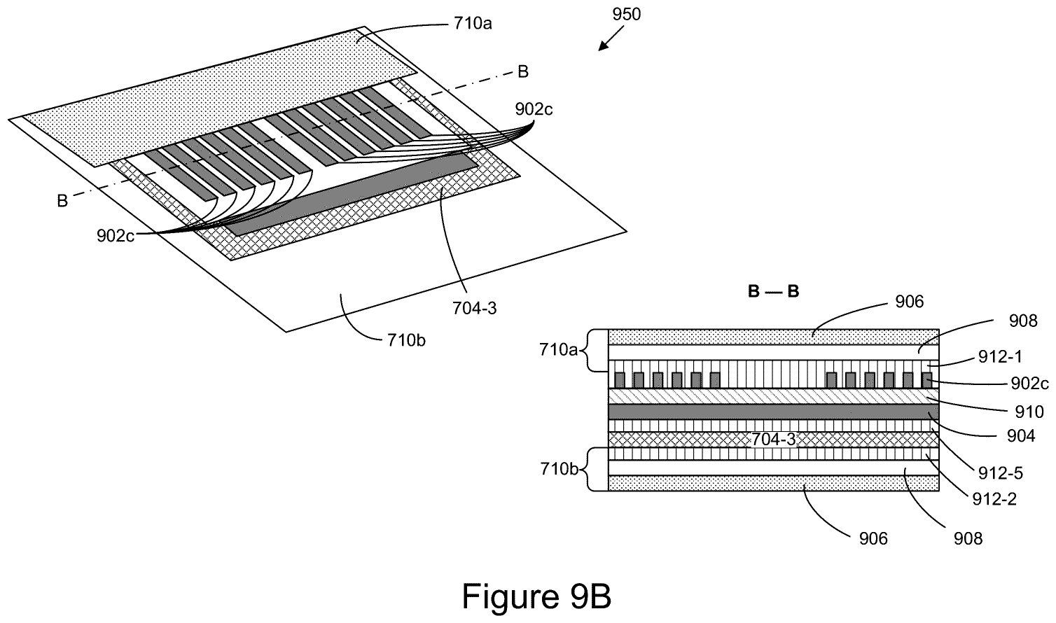

The fifth and sixth flex stack-up embodiments 700e and 700f include integrated strain-relief layers. In particular, the fifth flex stack-up embodiment 700e integrates a first integrated strain-relief layer 704-2 with a first unitary FPC layer 900a and cover layers 710 (FIG. 9A), while the sixth and 700f integrates a second integrated strain-relief layer 704-3 with a second unitary FPC layer 900b and cover layers 710 (FIG. 9B). The fifth and sixth flex stack-up embodiments 700e and 700f form unitary FPC assemblies as discussed below in reference to FIGS. 9A and 9B. The fifth and sixth flex stack-up embodiments 700e and 700f optimize neutral axis alignment, improve compatibility of stack-up embodiments with other devices, and increase robustness and reliability performance. Additional detail on the respective integrated strain-relief layers and respective unitary FPC layers is provide below in reference to FIGS. 9A and 9B. The fifth and sixth flex stack-up embodiments 700e and 700f further include a band overmold 702.

FIG. 8 illustrates one or more layers of a third example band portion 800 of the wearable device 100, in accordance with some embodiments. The third example band portion 800 includes the second flex stack-up embodiment 700b described above in reference to FIG. 7. The third example band portion 800 includes at least a strain-relief layer 803 (analogous with the strain-relief layers described above in reference to FIGS. 1A-7), an FPC layer 706-1, a wrapper 708-1 (which is formed of at least two layers, e.g., a first wrapper layer 708-1a and a second wrapper layer 708-1b), one or more wrapper adhesive layers 802, one or more receiver adhesive layers 804, one or more receiving structures 112, one or more AFEs 204, a LDA 806, one or more biopotential-signal sensing structures 104, a clasp retention element 808 (e.g., similar to an overmolded metal layer described above in reference to FIGS. 1A-7), a band overmold 702, and a textile 810. In some embodiments, the textile 810 is interchangeable with an elastomer overmold. The one or more receiving structures 112 are configured to couple with one or more AFEs 204.

The third example band portion 800 of the wearable device 100 shows formation of the wearable device 100. As shown by the third example band portion 800 of the wearable device 100, the strain-relief layer 803 is coupled, via a first adhesive layer (e.g., adhesive layers 506 and 606; FIGS. 5A and 6A), to the clasp retention element 808 (e.g., on a first surface of the strain-relief layer 803). The first wrapper layer 708-1a is coupled, via a second adhesive layer (e.g., adhesive layers 510 and 610; FIGS. 5A and 6A), over the strain-relief layer 803 (e.g., on a second surface of the strain-relief layer 803 opposite the first surface). The FPC layer 706-1 is couple on the first wrapper layer 708-1a via another adhesive layer (not shown).

As described above in reference to FIGS. 5A-7, the FPC layer 706-1 includes one or more portions on which the receiving structures 112 are coupled. Before the receiving structures 112 are coupled to the portions of the FPC layer 706-1, in some embodiments, the AFEs 204 are coupled on the portions of the FPC layer 706-1 via bonding or another adhesive layer. Similarly, respective wrapper adhesive layers 802 can be disposed over outer edges of the portions of the FPC layer 706-1 such that the second wrapper layer 708-1b can be disposed over the FPC layer 706- and couple with the first wrapper layer 708-1a. When the first and second wrapper layers 708-1a and 708-1b couple, the wrapper 708-1, which encloses the FPC layer 706-1, is formed. In some embodiments, the second wrapper layer 708-1b includes one or more cutouts such that the AFEs 204 are unwrapped. A wrapper (e.g., wrappers 708-1 and 708-2 described above in reference to FIG. 7) is configured to augment flex stiffness or decouple the midspan flex sections from a band portion to facilitate a tunnel approach. In addition, given that the wrappers establish a sealed channel, the band mold 702 can be optional.

After the wrapper 708-1 is formed, respective receiver adhesive layers 804 can be applied to portions of the wrapper 708-1 (e.g., edge portions of the wrapper 708-1 that outline receiver placement locations). The receiving structures 112 are coupled to the wrapper 708-1 via the respective receiver adhesive layers 804. As described above, the receiving structures 112 can receive and house the AFEs 204. The strain-relief layer 803, the clasp retention element 808, the wrapper 708-1, the FPC layer 706-1, and portions of the receiving structures 112 are encapsulated by the band overmold 702. In some embodiments, a textile is disposed over the band mold 702. In some embodiments, the LDA 806 can be applied to the receiving structures 112 before the biopotential-signal sensing structures 104 are coupled thereto. In some embodiments, the LDA 806 is an adhesive or sealant.

FIGS. 9A-9B illustrate cross-sectional views of integrated strain-relief layers, in accordance with some embodiments. FIG. 9A shows a first integrated strain-relief layer (e.g., a first unitary FPC layer 900) analogous with the fifth flex stack-up embodiment 700e (FIG. 7) and FIG. 9B shows a second integrated strain-relief layer (e.g., a second unitary FPC layer 950) analogous with the sixth flex stack-up embodiment 700f (FIG. 7).

The first unitary FPC layer 900 includes a strain-relief layer 704-2 embedded between conductive portions. Specifically, the strain-relief layer 704-2 is embedded between a first set of copper traces 902a and a second set of copper traces 902b. The conductive portions and the strain-relief layer 704-2 form a core region of the first unitary FPC layer 900. The core region is coupled between at least two cover layers 710. In particular, the core region is sandwiched between a first cover layer 710a and a second cover layer 710b. The at least two cover layers 710 provide electromagnetic interference (EMI) protection. In some embodiments, adhesive layers 912 couple the at least two cover layers 710 to the core region.

A cross-sectional view of a cut through A-A along a widthwise portion of the first unitary FPC layer 900 illustrates one or more layers of the first unitary FPC layer 900. The strain-relief layer 704-2 is positioned at the center of the first unitary FPC layer 900. The strain-relief layer 704-2 is disposed between at least two conductive portions. Each conductive portion includes copper traces 902, a polyimide base layer 910, and a copper base layer 904. In some embodiments, the at least two conductive portions are coupled to the strain-relief layer 704-2 via one or more adhesive layers 912-3 and 912-4. The strain-relief layer 704-2, the at least two conductive portions, and the one or more adhesive layers 912-3 and 912-4 define a widthwise portion of the first unitary FPC layer 900.

The strain-relief layer 704-2 and the at least two conductive portions are disposed between at least two cover layers 710a and 710b. The strain-relief layer 704-2, the at least two conductive portions, and the at least two cover layers 710a and 710b define a thickness of the first unitary FPC layer 900. Each cover layer 710 includes an EMI protection layer 906 (e.g., configured to shield electromagnetic interference), a polyimide coverlay layer 908, and an adhesive layer 912 (e.g., 912-1 or 912-2). In some embodiments, the adhesive layers 912 of the cover layers 710 form the adhesive layers (e.g., 912-3 and 912-4) between the strain-relief layer 704-2 and the at least two conductive portions.

In some embodiments, each cover layer 710 has a thickness of approximately 50 μm (e.g., +/−10 μm). Each layer of the cover layer 710 can have a respective thickness. For example, the EMI protective layer 906 can have a thickness of approximately 8 μm (e.g., +/−2 μm), the polyimide coverlay layer 908 can have a thickness of approximately 12.5 μm (e.g., +/−5 μm), and the adhesive layer 912 can have a thickness of approximately 12 μm (e.g., +/−5 to 10 μm). The strain-relief layer 704-2 and the conductive portions have a thickness of approximately 35 μm (e.g., +/−5 μm). Each layer of the conductive portions can have a respective thickness. For example, the copper traces 902 can have a thickness of approximately 10 μm thick (e.g., +/−3 μm), the polyimide base layer 910 can have a thickness of approximately 12 μm (e.g., +/−3 μm), and the copper base layer 904 can have a thickness of approximately 10 μm (e.g., +/−3 μm). As the skilled artisan will appreciate upon reading the descriptions provided herein, the actual thickness of each layer can vary, subject to design specifications and manufacturing tolerances in an assembly process.

The second unitary FPC layer 950 includes a strain-relief layer 704-3 disposed between at least two cover layers 710 and a distinct conductive portion disposed on a surface of the strain-relief layer 704-3 (e.g., one side of the strain-relief layer 704-3). The distinct conductive portion includes a third set of copper traces 902c. The strain-relief layer 704-3 and the conductive portion form a distinct core region of the second unitary FPC layer 950. As described above in reference to the first unitary FPC layer 900, the at least two cover layers 710 provide electromagnetic interference (EMI) protection. In some embodiments, adhesive layers 912 couple the at least two cover layers 710 to the distinct core region.

A cross-sectional view of a cut through B-B along a widthwise portion of the second unitary FPC layer 950 illustrates one or more layers of the second unitary FPC layer 950. The strain-relief layer 704-3 is disposed over the second cover layer 710b and extends the widthwise portion of the second unitary FPC layer 950. The distinct conductive portion is disposed over the strain-relief layer 704-3 (on a surface opposite the surface coupled to the second cover layer 710b). The distinct conductive portion includes at least the third set of copper traces 902c, the polyimide base layer 910, and the copper base layer 904. An additional adhesive layer 912-5 can be disposed between the distinct conductive portion and the strain-relief layer 704-3 to couple the two layers. A first cover layer 710a is disposed above the distinct conductive portion.

The second unitary FPC layer 950 has a thickness substantially similar to the first unitary FPC layer 900. For example, the cover layers 710, the distinct conductive portion, the strain-relief layer 704-3, and their respective individual layers have the same thicknesses to those described above in reference to FIG. 9A. The additional adhesive layer 912-5 has a thickness of approximately 12 μm (e.g., +/−5 μm). As the skilled artisan will appreciate upon reading the descriptions provided herein, the actual thickness of each layer can vary, subject to design specifications and manufacturing tolerances in an assembly process.

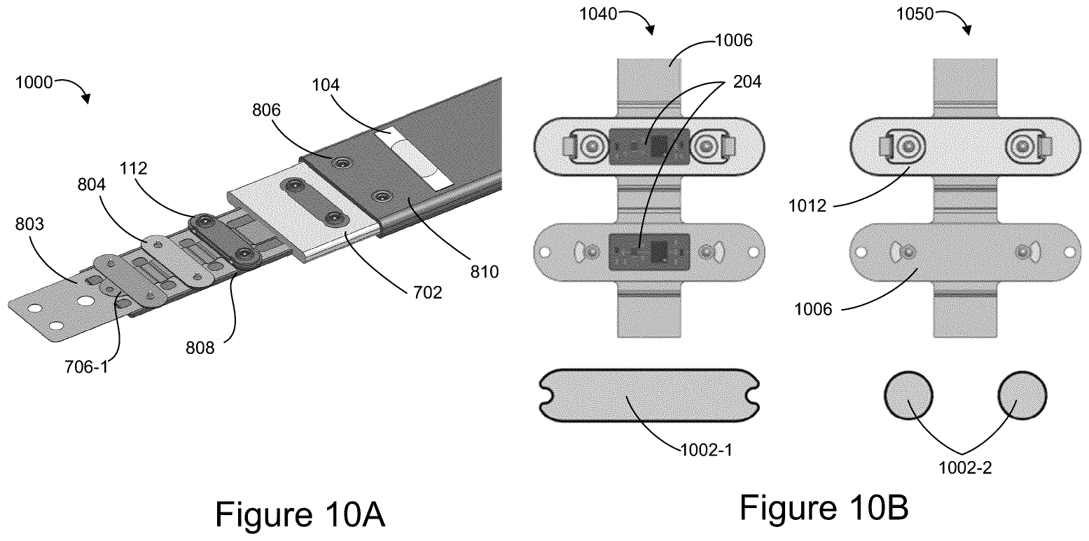

FIG. 10A illustrates an AFE offloaded wearable device 1000, in accordance with some embodiments. The AFE offloaded wearable device 1000 removes the AFEs from the mid band. The functions performed by the AFEs are performed by a computer system (e.g., computer system 1460; FIGS. 14A and 14B). The AFE offloaded wearable device 1000 is analogous with the third example band portion 800 described above in reference to FIG. 8. For example, the AFE offloaded wearable device 1000 includes at least a strain-relief layer 803, an FPC layer 706-1, one or more receiver adhesive layers 804, one or more receiving structures 112, a LDA 806, one or more biopotential-signal sensing structures 104, a clasp retention element 808, a band overmold 702, and a textile 810.

The AFE offloaded wearable device 1000 reduces constraints and yield loss in overmolding due to AFE damage. Additionally, the AFE offloaded wearable device 1000 allows strain-relief layer 803 to mate with the FPC layer 706-1 to further align neutral axis to the traces. Additionally, the AFE offloaded wearable device 1000 optimizes receiver to TPSiV bonding. The AFE offloaded wearable device 1000 further allows for flexible biopotential-signal sensing structures 104. Further, by offloading the AFEs from the mid band to compute core, additional space at portions under the biopotential-signal sensing structure 104 becomes available, which further alleviates stiffness and ingress due to surface-mount technology components in the band portion.

FIG. 10B illustrates AFEs removed from an FPC layer, in accordance with some embodiments. A first example FPC layer portion 1040 shows one or more AFEs 204 coupled with the FPC layer 1006 (similar with FPC layers described above in reference to FIGS. 1A-10A). Due to the additional constraints imposed by AFEs 204, additional stiffener 1002-1 is needed. Alternatively, by offloading the AFEs 204, as shown in a second example FPC layer portion 1050, a distinct stiffener 1002-2 is needed (e.g., stiffener for receiver structure mounting surfaces 1012).

FIG. 11 illustrates an alternate band portion of a wearable device, in accordance with some embodiments. The alternate band portion 1100 includes a micro-coax assembly 1112 (e.g., one or more micro-coaxial wires), one or more drain wires 1110, one or more termination elements 1104, one or more ground plates 1106, a low pressure molding 1108, a strain-relief layer 1102 (similar to the strain-relief layer described above in reference to FIGS. 1A-10), a clasp retention element 808, one or more biopotential-signal sensing structures 104, a band mold 702, and a textile 810. The micro-coax assembly 1112 and the drain wires 1110 are disposed on the strain-relief layer 1102 (e.g., on the same surface). The micro-coax assembly 1112 and the drain wires 1110 are configured to couple with one or more termination elements 1104 and/or one or more ground plates 1106. In particular, the micro-coaxial wires 1112 can be terminated through retention plates and solders at ring terminals of the termination elements 1104. Shield layers of the drain wires 1110 and/or micro-coaxial wires 1112 can be exposed and bundled at each bracket for common grounding. The micro-coax assembly 1112, the drain wires 1110, ground plates 1106, and the termination elements 1104 are used in place of an FPC layer (described above in reference to FIGS. 1A-10), such that the micro-coax assembly 1112 and the drain wires 1110 communicatively couple with a computer system (e.g., computer system 1460; FIGS. 14A and 14B). The micro-coax assembly 1112 is configured to reduced cross-talk, enhance robustness and flexibility, lower signal losses, improve impedance matching, and improve miniaturization. Further, the low pressure molding 1108 is configured for ingress protection and joint strain relief.

The strain-relief layer 1102, the one or more biopotential-signal sensing structures 104, the band mold 702, and the textile 810 are analogous to corresponding components described above in reference to FIGS. 1A-10.

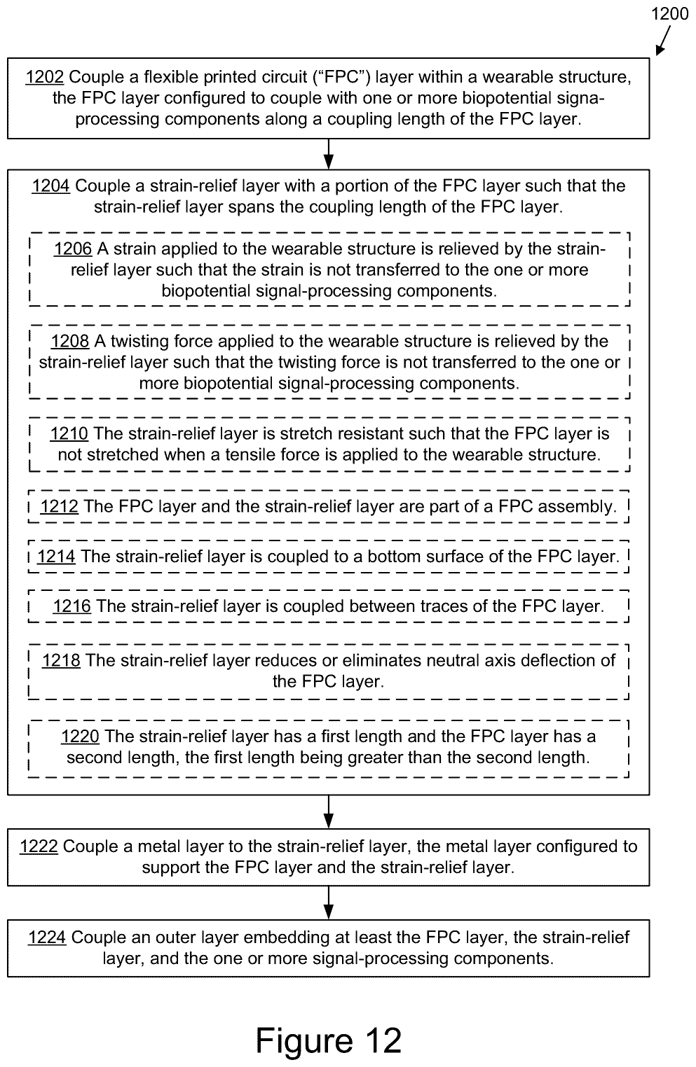

FIG. 12 illustrates a flow diagram of an example method of manufacturing a wearable structure of a wearable device, in accordance with some embodiments. Specifically, the flow diagram of FIG. 12 can be used to manufacture a wearable structure of the embodiments described above in reference to FIGS. 1A-11. In some embodiments, the various operations of the methods described herein are interchangeable and/or optional, and respective operations of the methods are performed by any of the aforementioned devices, systems, or combination of devices and/or systems. For convenience, the method operations will be described below as being performed by particular component or device, but should not be construed as limiting the performance of the operation to the particular device in all embodiments.

(A1) The method of 1200 includes coupling (1202) an FPC layer within a wearable structure. The FPC layer is configured to couple with one or more biopotential signa-processing components along a coupling length of the FPC layer (e.g., a lengthwise portion of a band portion). FIGS. 1A-11 provide examples of different embodiments of an FPC layer and coupling locations of the one or more biopotential signa-processing components. The method 1200 includes coupling (1204) a strain-relief layer with a portion of the FPC layer such that the strain-relief layer spans the coupling length of the flexible printed circuit layer. For example, as shown and described above in reference to FIGS. 1A-11, a strain-relief layer extends the lengthwise portion of a band portion. In some embodiments, the method 1200 includes coupling (1222) a metal layer to the strain-relief layer. The metal layer configured to support the FPC layer and the strain-relief layer. In some embodiments, the metal layer is used as a clasp retention mechanism (e.g., a surface for coupling a magnet that allows a user to adjust a size of the wearable structure). Different examples of the metal layer are provided above in reference to at least FIGS. 4, 5A, 6A, 8, 10A, and 11 (e.g., overmolded metal layer). The method 1200 further includes coupling (1224) an outer layer embedding at least the FPC layer, the strain-relief layer, and the one or more signal-processing components. For example, as shown and described above in reference to FIGS. 1A-11, a textile and/or elastomer overmold can encase one or more components of a wearable structure.

(A2) In some embodiments of A1, a strain applied to the wearable structure is relieved (1206) by the strain-relief layer such that the strain is not transferred to the one or more biopotential signal-processing components.

(A3) In some embodiments of A1-A2, a twisting force applied to the wearable structure is relieved (1208) by the strain-relief layer such that the twisting force is not transferred to the one or more biopotential signal-processing components (e.g., AFEs 204; FIG. 2).

(A4) In some embodiments of A1-A3, the strain-relief layer is (1210) stretch resistant such that the FPC layer in not stretched when a tensile force is applied to the wearable structure.

(A5) In some embodiments of A1-A4, one or more biopotential signal-processing components are electrically coupled with one or more biopotential-signal-sensing electrodes (e.g., biopotential-signal sensing structures 104; FIG. 1A).

(A6) In some embodiments of A1-A5, the FPC layer and the strain-relief layer are (1212) part of a FPC assembly.

(A7) In some embodiments of A1-A6, the strain-relief layer is coupled (1214) to a bottom surface of the flexible printed circuit layer. For example, as described above in at least FIGS. 5A and 6A, the flexible printed circuit layer is disposed above a respective strain-relief layer.

(A8) In some embodiments of A1-A7, the strain-relief layer is coupled (1216) between traces of the flexible printed circuit layer. For example, as shown in FIG. 9A, the strain-relief layer can be disposed between a first set of copper traces 902a and a second set of copper traces 902b.

(A9) In some embodiments of A1-A8, the strain-relief layer reduces (1218) or eliminates neutral axis deflection of the FPC layer.

(A10) In some embodiments of A1-A9, the strain-relief layer has (1220) a first length and the FPC layer has a second length, first length being greater than the second length. Different examples of the flexible printed circuit layer and the strain-relief layers are provided above in reference to FIGS. 5A-7.

(A11) In some embodiments of A1-A10, the strain-relief layer does not stretch.

(A12) In some embodiments of A1-A11, the strain-relief layer is formed of Vectran, Kevlar, or other polymers.

(A13) In some embodiments of A1-A12, the wearable structure is at least one of a wrist-wearable device, a head-wearable device, or a wearable garment.

(A14) In some embodiments of A1-A13, the strain-relief layer has a predetermined thickness.

(A15) In some embodiments of A1-A14, the wearable structure is configured to couple with a compute core and the compute core is configured to communicatively couple with the FPC layer. The compute core is an example of a computer system (e.g., computer system 1460; FIGS. 14A and 14B).

The devices described above are further detailed below, including systems, wrist-wearable devices, and headset devices. Specific operations described above may occur as a result of specific hardware, such hardware is described in further detail below. The devices described below are not limiting and features on these devices can be removed or additional features can be added to these devices. The different devices can include one or more analogous hardware components. For brevity, analogous devices and components are described below. Any differences in the devices and components are described below in their respective sections.

As described herein, a processor (e.g., a central processing unit (CPU) or microcontroller unit (MCU)), is an electronic component that is responsible for executing instructions and controlling the operation of an electronic device (e.g., a wrist-wearable device 1400, a head-wearable device 1360, an HIPD 1370, or other computer system). There are various types of processors that may be used interchangeably or specifically required by embodiments described herein. For example, a processor may be (i) a general processor designed to perform a wide range of tasks, such as running software applications, managing operating systems, and performing arithmetic and logical operations; (ii) a microcontroller designed for specific tasks such as controlling electronic devices, sensors, and motors; (iii) a graphics processing unit (GPU) designed to accelerate the creation and rendering of images, videos, and animations (e.g., virtual-reality animations, such as three-dimensional modeling); (iv) a field-programmable gate array (FPGA) that can be programmed and reconfigured after manufacturing and/or customized to perform specific tasks, such as signal processing, cryptography, and machine learning; (v) a digital signal processor (DSP) designed to perform mathematical operations on signals such as audio, video, and radio waves. One of skill in the art will understand that one or more processors of one or more electronic devices may be used in various embodiments described herein.

As described herein, controllers are electronics components that manage and coordinate the operation of other components within an electronic device (e.g., controlling inputs, processing data, and/or generating outputs). Examples of controllers can include (i) microcontrollers, including small, low-power controllers that are commonly used in embedded systems and Internet of Things (IoT) devices; (ii) programmable logic controllers (PLCs) that may be configured to be used in industrial automation systems to control and monitor manufacturing processes; (iii) system-on-a-chip (SoC) controllers that integrate multiple components such as processors, memory, I/O interfaces, and other peripherals into a single chip; and/or DSPs. As described herein, a graphics module is a component or software module that is designed to handle graphical operations and/or processes, and can include a hardware module and/or a software module.

As described herein, memory refers to electronics components in a computer or electronic device that store data and instructions for the processor to access and manipulate. The devices described herein can include volatile and non-volatile memory. Examples of memory can include (i) random access memory (RAM), such as DRAM, SRAM, DDR RAM or other random access solid state memory devices, configured to store data and instructions temporarily; (ii) read-only memory (ROM) configured to store data and instructions permanently (e.g., one or more portions of system firmware and/or boot loaders); (iii) flash memory, magnetic disk storage devices, optical disk storage devices, other non-volatile solid state storage devices, which can be configured to store data in electronic devices (e.g., universal serial bus (USB) drives, memory cards, and/or solid-state drives (SSDs)); and (iv) cache memory configured to temporarily store frequently accessed data and instructions. Memory, as described herein, can include structured data (e.g., SQL databases, MongoDB databases, GraphQL data, or JSON data). Other examples of memory can include: (i) profile data, including user account data, user settings, and/or other user data stored by the user; (ii) sensor data detected and/or otherwise obtained by one or more sensors; (iii) media content data including stored image data, audio data, documents, and the like; (iv) application data, which can include data collected and/or otherwise obtained and stored during use of an application; and/or any other types of data described herein.

As described herein, a power system of an electronic device is configured to convert incoming electrical power into a form that can be used to operate the device. A power system can include various components, including (i) a power source, which can be an alternating current (AC) adapter or a direct current (DC) adapter power supply; (ii) a charger input that can be configured to use a wired and/or wireless connection (which may be part of a peripheral interface, such as a USB, micro-USB interface, near-field magnetic coupling, magnetic inductive and magnetic resonance charging, and/or radio frequency (RF) charging); (iii) a power-management integrated circuit, configured to distribute power to various components of the device and ensure that the device operates within safe limits (e.g., regulating voltage, controlling current flow, and/or managing heat dissipation); and/or (iv) a battery configured to store power to provide usable power to components of one or more electronic devices.

As described herein, peripheral interfaces are electronics components (e.g., of electronic devices) that allow electronic devices to communicate with other devices or peripherals and can provide a means for input and output of data and signals. Examples of peripheral interfaces can include (i) USB and/or micro-USB interfaces configured for connecting devices to an electronic device; (ii) Bluetooth interfaces configured to allow devices to communicate with each other, including Bluetooth low energy (BLE); (iii) near-field communication (NFC) interfaces configured to be short-range wireless interfaces for operations such as access control; (iv) POGO pins, which may be small, spring-loaded pins configured to provide a charging interface; (v) wireless charging interfaces; (vi) global-position system (GPS) interfaces; (vii) Wi-Fi interfaces for providing a connection between a device and a wireless network; and (viii) sensor interfaces.

As described herein, sensors are electronics components (e.g., in and/or otherwise in electronic communication with electronic devices, such as wearable devices) configured to detect physical and environmental changes and generate electrical signals. Examples of sensors can include (i) imaging sensors for collecting imaging data (e.g., including one or more cameras disposed on a respective electronic device); (ii) biopotential-signal sensors; (iii) inertial measurement unit (e.g., IMUs) for detecting, for example, angular rate, force, magnetic field, and/or changes in acceleration; (iv) heart rate sensors for measuring a user's heart rate; (v) SpO2 sensors for measuring blood oxygen saturation and/or other biometric data of a user; (vi) capacitive sensors for detecting changes in potential at a portion of a user's body (e.g., a sensor-skin interface) and/or the proximity of other devices or objects; and (vii) light sensors (e.g., ToF sensors, infrared light sensors, or visible light sensors), and/or sensors for sensing data from the user or the user's environment. As described herein biopotential-signal-sensing components are devices used to measure electrical activity within the body (e.g., biopotential-signal sensors). Some types of biopotential-signal sensors include: (i) electroencephalography (EEG) sensors configured to measure electrical activity in the brain to diagnose neurological disorders; (ii) electrocardiography (ECG or EKG) sensors configured to measure electrical activity of the heart to diagnose heart problems; (iii) electromyography (EMG) sensors configured to measure the electrical activity of muscles and diagnose neuromuscular disorders; (iv) electrooculography (EOG) sensors configured to measure the electrical activity of eye muscles to detect eye movement and diagnose eye disorders.

As described herein, an application stored in memory of an electronic device (e.g., software) includes instructions stored in the memory. Examples of such applications include (i) games; (ii) word processors; (iii) messaging applications; (iv) media-streaming applications; (v) financial applications; (vi) calendars; (vii) clocks; (viii) web browsers; (ix) social media applications, (x) camera applications, (xi) web-based applications; (xii) health applications; (xiii) artificial-reality (AR) applications, and/or any other applications that can be stored in memory. The applications can operate in conjunction with data and/or one or more components of a device or communicatively coupled devices to perform one or more operations and/or functions.

As described herein, communication interface modules can include hardware and/or software capable of data communications using any of a variety of custom or standard wireless protocols (e.g., IEEE 802.15.4, Wi-Fi, ZigBee, 6LoWPAN, Thread, Z-Wave, Bluetooth Smart, ISA100.11a, WirelessHART, or MiWi), custom or standard wired protocols (e.g., Ethernet or HomePlug), and/or any other suitable communication protocol, including communication protocols not yet developed as of the filing date of this document. A communication interface is a mechanism that enables different systems or devices to exchange information and data with each other, including hardware, software, or a combination of both hardware and software. For example, a communication interface can refer to a physical connector and/or port on a device that enables communication with other devices (e.g., USB, Ethernet, HDMI, or Bluetooth). In some embodiments, a communication interface can refer to a software layer that enables different software programs to communicate with each other (e.g., application programming interfaces (APIs) and protocols such as HTTP and TCP/IP).

As described herein, a graphics module is a component or software module that is designed to handle graphical operations and/or processes, and can include a hardware module and/or a software module.

As described herein, non-transitory computer-readable storage media are physical devices or storage medium that can be used to store electronic data in a non-transitory form (e.g., such that the data is stored permanently until it is intentionally deleted or modified).

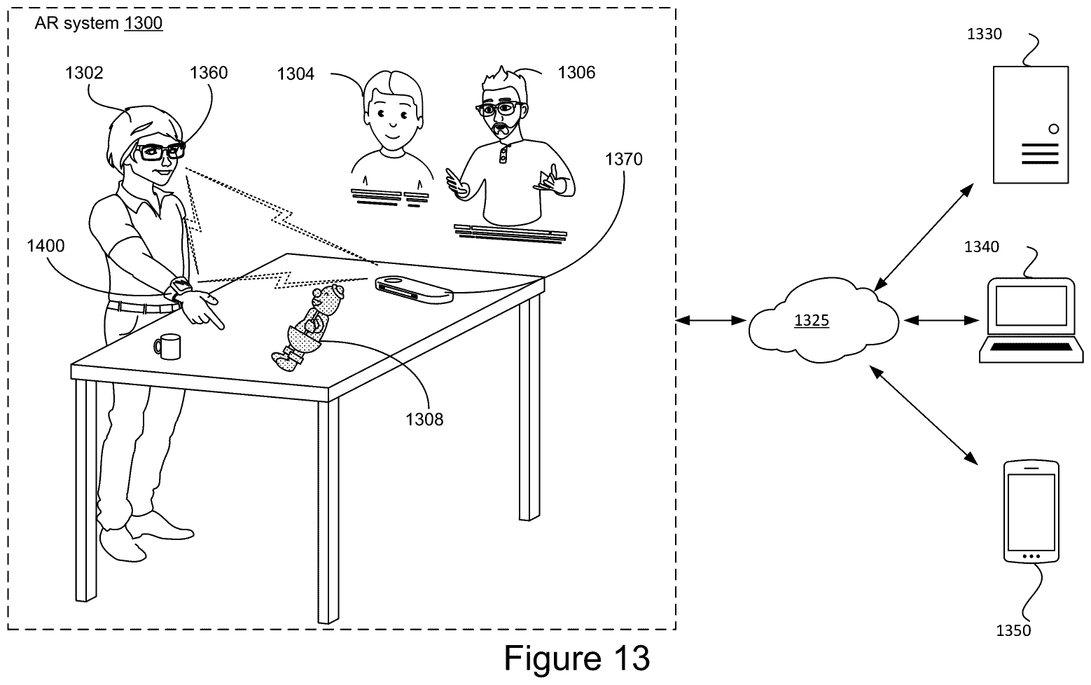

FIG. 13 illustrates an example artificial-reality system, in accordance with some embodiments. FIG. 13 shows an AR system 1300 and example user interactions using a wrist-wearable device 1400, a head-wearable device (e.g., AR device 1360), and/or a handheld intermediary processing device (HIPD) 1370.

The wrist-wearable device 1400 and its constituent components are described below in reference to FIGS. 14A-14B. The wrist-wearable device 1400, the head-wearable devices, and/or the HIPD 1370 can communicatively couple via a network 1325 (e.g., cellular, near field, Wi-Fi, personal area network, wireless LAN, etc.). Additionally, the wrist-wearable device 1400, the head-wearable devices, and/or the HIPD 1370 can also communicatively couple with one or more servers 1330, computers 1340 (e.g., laptops, computers, etc.), mobile devices 1350 (e.g., smartphones, tablets, etc.), and/or other electronic devices via the network 1325 (e.g., cellular, near field, Wi-Fi, personal area network, wireless LAN, etc.)

In FIG. 13, a user 1402 is shown wearing the wrist-wearable device 1400 and the AR device 1360, and having the HIPD 1370 on their desk. The wrist-wearable device 1400, the AR device 1360, and the HIPD 1370 facilitate user interaction with an AR environment. In particular, as shown by the AR system 1400, the wrist-wearable device 1400, the AR device 1360, and/or the HIPD 1370 cause presentation of one or more avatars 1304, digital representations of contacts 1306, and virtual objects 1308. As discussed below, the user 1302 can interact with the one or more avatars 1304, digital representations of the contacts 1306, and virtual objects 1308 via the wrist-wearable device 1400, the AR device 1360, and/or the HIPD 1370.

The user 1302 can use any of the wrist-wearable device 1400, the AR device 1360, and/or the HIPD 1370 to provide user inputs. For example, the user 1302 can perform one or more hand gestures that are detected by the wrist-wearable device 1400 (e.g., using one or more EMG sensors and/or IMUs, described below in reference to FIGS. 14A-14B) and/or AR device 1360 (e.g., using one or more image sensors or cameras) to provide a user input. Alternatively, or additionally, the user 1302 can provide a user input via one or more touch surfaces of the wrist-wearable device 1400, the AR device 1360, and/or the HIPD 1370, and/or voice commands captured by a microphone of the wrist-wearable device 1400, the AR device 1360, and/or the HIPD 1370. In some embodiments, the wrist-wearable device 1400, the AR device 1360, and/or the HIPD 1370 include a digital assistant to help the user in providing a user input (e.g., completing a sequence of operations, suggesting different operations or commands, providing reminders, confirming a command). In some embodiments, the user 1302 can provide a user input via one or more facial gestures and/or facial expressions. For example, cameras of the wrist-wearable device 1400, the AR device 1360, and/or the HIPD 1370 can track the user 1302's eyes for navigating a user interface.

The wrist-wearable device 1400, the AR device 1360, and/or the HIPD 1370 can operate alone or in conjunction to allow the user 1302 to interact with the AR environment. In some embodiments, the HIPD 1370 is configured to operate as a central hub or control center for the wrist-wearable device 1400, the AR device 1360, and/or another communicatively coupled device. For example, the user 1302 can provide an input to interact with the AR environment at any of the wrist-wearable device 1400, the AR device 1360, and/or the HIPD 1370, and the HIPD 1370 can identify one or more back-end and front-end tasks to cause the performance of the requested interaction and distribute instructions to cause the performance of the one or more back-end and front-end tasks at the wrist-wearable device 1400, the AR device 1360, and/or the HIPD 1370. In some embodiments, a back-end task is a background-processing task that is not perceptible by the user (e.g., rendering content, decompression, compression, etc.), and a front-end task is a user-facing task that is perceptible to the user (e.g., presenting information to the user, providing feedback to the user, etc.)). The HIPD 1370 can perform the back-end tasks and provide the wrist-wearable device 1400 and/or the AR device 1360 operational data corresponding to the performed back-end tasks such that the wrist-wearable device 1400 and/or the AR device 1360 can perform the front-end tasks. In this way, the HIPD 1370, which has more computational resources and greater thermal headroom than the wrist-wearable device 1400 and/or the AR device 1360, performs computationally intensive tasks and reduces the computer resource utilization and/or power usage of the wrist-wearable device 1400 and/or the AR device 1360.

In the example shown by the AR system 1300, the HIPD 1370 identifies one or more back-end tasks and front-end tasks associated with a user request to initiate an AR video call with one or more other users (represented by the avatar 1304 and the digital representation of the contact 1306) and distributes instructions to cause the performance of the one or more back-end tasks and front-end tasks. In particular, the HIPD 1370 performs back-end tasks for processing and/or rendering image data (and other data) associated with the AR video call and provides operational data associated with the performed back-end tasks to the AR device 1360 such that the AR device 1360 performs front-end tasks for presenting the AR video call (e.g., presenting the avatar 1304 and the digital representation of the contact 1306).

In some embodiments, the HIPD 1370 can operate as a focal or anchor point for causing the presentation of information. This allows the user 1302 to be generally aware of where information is presented. For example, as shown in the AR system 1300, the avatar 1304 and the digital representation of the contact 1306 are presented above the HIPD 1370. In particular, the HIPD 1370 and the AR device 1360 operate in conjunction to determine a location for presenting the avatar 1304 and the digital representation of the contact 1306. In some embodiments, information can be presented within a predetermined distance from the HIPD 1370 (e.g., within five meters). For example, as shown in the AR system 1300, the virtual object 1308 is presented on the desk some distance from the HIPD 1370. Similar to the above example, the HIPD 1370 and the AR device 1360 can operate in conjunction to determine a location for presenting the virtual object 1308. Alternatively, in some embodiments, presentation of information is not bound by the HIPD 1370. More specifically, the avatar 1304, the digital representation of the contact 1306, and the virtual object 1308 do not have to be presented within a predetermined distance of the HIPD 1370.

User inputs provided at the wrist-wearable device 1400, the AR device 1360, and/or the HIPD 1370 are coordinated such that the user can use any device to initiate, continue, and/or complete an operation. For example, the user 1302 can provide a user input to the AR device 1360 to cause the AR device 1360 to present the virtual object 1308 and, while the virtual object 1308 is presented by the AR device 1360, the user 1302 can provide one or more hand gestures via the wrist-wearable device 1400 to interact and/or manipulate the virtual object 1308.

Having discussed an example AR system, the wrist-wearable device 1400 for interacting with such an AR system, and other computing systems more generally, will now be discussed in greater detail below. Some definitions of devices and components that can be included in some or all of the example devices discussed below are defined here for ease of reference. A skilled artisan will appreciate that certain types of the components described below may be more suitable for a particular set of devices, and less suitable for a different set of devices. But subsequent reference to the components defined here should be considered to be encompassed by the definitions provided.

In some embodiments discussed below example devices and systems, including electronic devices and systems, will be discussed. Such example devices and systems are not intended to be limiting, and one of skill in the art will understand that alternative devices and systems to the example devices and systems described herein may be used to perform the operations and construct the systems and device that are described herein.

As described herein, an electronic device is a device that uses electrical energy to perform a specific function. It can be any physical object that contains electronics components such as transistors, resistors, capacitors, diodes, and integrated circuits. Examples of electronic devices include smartphones, laptops, digital cameras, televisions, gaming consoles, and music players, as well as the example electronic devices discussed herein. As described herein, an intermediary electronic device is a device that sits between two other electronic devices, and/or a subset of components of one or more electronic devices and facilitates communication, and/or data processing and/or data transfer between the respective electronic devices and/or electronics components.

### Example Wrist-Wearable Devices

FIGS. 14A and 14B illustrate an example wrist-wearable device 1400, in accordance with some embodiments. The wrist-wearable device 1400 is an instance of the wearable device described in reference to FIGS. 1A-12 herein, such that the wrist-wearable devices of FIGS. 1A-12 should be understood to have the features of the wrist-wearable device 1400 and vice versa. FIG. 14A illustrates components of the wrist-wearable device 1400, which can be used individually or in combination, including combinations that include other electronic devices and/or electronics components.

FIG. 14A shows a wearable band 1410 and a watch body 1420 (or capsule) being coupled, as discussed below, to form the wrist-wearable device 1400. The wrist-wearable device 1400 can perform various functions and/or operations associated with navigating through user interfaces and selectively opening applications.

As will be described in more detail below, operations executed by the wrist-wearable device 1400 can include (i) presenting content to a user (e.g., displaying visual content via a display 1405); (ii) detecting (e.g., sensing) user input (e.g., sensing a touch on peripheral button 1423 and/or at a touch screen of the display 1405, a hand gesture detected by sensors (e.g., biopotential sensors)); (iii) sensing biometric data via one or more sensors 1413 (e.g., neuromuscular signals, heart rate, temperature, sleep, etc.); messaging (e.g., text, speech, video, etc.); image capture via one or more imaging devices or cameras 1425; wireless communications (e.g., cellular, near field, Wi-Fi, personal area network, etc.); location determination; financial transactions; providing haptic feedback; alarms; notifications; biometric authentication; health monitoring; sleep monitoring.

The above-example functions can be executed independently in the watch body 1420, independently in the wearable band 1410, and/or via an electronic communication between the watch body 1420 and the wearable band 1410. In some embodiments, functions can be executed on the wrist-wearable device 1400 while an AR environment is being presented (e.g., the AR system 1300). As the skilled artisan will appreciate upon reading the descriptions provided herein, the novel wearable devices described herein can be used with other types of AR environments.

The wearable band 1410 can be configured to be worn by a user such that an inner (or inside) surface of the wearable structure 1411 of the wearable band 1410 is in contact with the user's skin. When worn by a user, sensors 1413 contact the user's skin. The sensors 1413 can sense biometric data such as a user's heart rate, saturated oxygen level, temperature, sweat level, neuromuscular signal sensors, or a combination thereof. The sensors 1413 can also sense data about a user's environment, including a user's motion, altitude, location, orientation, gait, acceleration, position, or a combination thereof. In some embodiments, the sensors 1413 are configured to track a position and/or motion of the wearable band 1410. The one or more sensors 1413 can include any of the sensors defined above and/or discussed below with respect to FIG. 14B.

The one or more sensors 1413 can be distributed on an inside and/or an outside surface of the wearable band 1410. In some embodiments, the one or more sensors 1413 are uniformly spaced along the wearable band 1410. Alternatively, in some embodiments, the one or more sensors 1413 are positioned at distinct points along the wearable band 1410. As shown in FIG. 14A, the one or more sensors 1413 can be the same or distinct. For example, in some embodiments, the one or more sensors 1413 can be shaped as a pill (e.g., sensor 1413a), an oval, a circle a square, an oblong (e.g., sensor 1413c) and/or any other shape that maintains contact with the user's skin (e.g., such that neuromuscular signal and/or other biometric data can be accurately measured at the user's skin). In some embodiments, the one or more sensors 1413 are aligned to form pairs of sensors (e.g., for sensing neuromuscular signals based on differential sensing within each respective sensor). For example, sensor 1413b is aligned with an adjacent sensor to form sensor pair 1414a and sensor 1413d is aligned with an adjacent sensor to form sensor pair 1414b. In some embodiments, the wearable band 1410 does not have a sensor pair. Alternatively, in some embodiments, the wearable band 1410 has a predetermined number of sensor pairs (one pair of sensors, three pairs of sensors, four pairs of sensors, six pairs of sensors, sixteen pairs of sensors, etc.).

The wearable band 1410 can include any suitable number of sensors 1413. In some embodiments, the number and arrangements of sensors 1413 depend on the particular application for which the wearable band 1410 is used. For instance, a wearable band 1410 configured as an armband, wristband, or chest-band may include a plurality of sensors 1413 with different number of sensors 1413 and different arrangement for each use case, such as medical use cases, compared to gaming or general day-to-day use cases.

In accordance with some embodiments, the wearable band 1410 further includes an electrical ground electrode and a shielding electrode. The electrical ground and shielding electrodes, like the sensors 1413, can be distributed on the inside surface of the wearable band 1410 such that they contact a portion of the user's skin. For example, the electrical ground and shielding electrodes can be at an inside surface of coupling mechanism 1416 or an inside surface of a wearable structure 1411. The electrical ground and shielding electrodes can be formed and/or use the same components as the sensors 1413. In some embodiments, the wearable band 1410 includes more than one electrical ground electrode and more than one shielding electrode.

The sensors 1413 can be formed as part of the wearable structure 1411 of the wearable band 1410. In some embodiments, the sensors 1413 are flush or substantially flush with the wearable structure 1411 such that they do not extend beyond the surface of the wearable structure 1411. While flush with the wearable structure 1411, the sensors 1413 are still configured to contact the user's skin (e.g., via a skin-contacting surface). Alternatively, in some embodiments, the sensors 1413 extend beyond the wearable structure 1411 a predetermined distance (e.g., 0.1 mm to 2 mm) to make contact and depress into the user's skin. In some embodiments, the sensors 1413 are coupled to an actuator (not shown) configured to adjust an extension height (e.g., a distance from the surface of the wearable structure 1411) of the sensors 1413 such that the sensors 1413 make contact and depress into the user's skin. In some embodiments, the actuators adjust the extension height between 0.01 mm to 1.2 mm. This allows the user to customize the positioning of the sensors 1413 to improve the overall comfort of the wearable band 1410 when worn while still allowing the sensors 1413 to contact the user's skin. In some embodiments, the sensors 1413 are indistinguishable from the wearable structure 1411 when worn by the user.

The wearable structure 1411 can be formed of an elastic material, elastomers, etc., configured to be stretched and fitted to be worn by the user. In some embodiments, the wearable structure 1411 is a textile or woven fabric. As described above, the sensors 1413 can be formed as part of a wearable structure 1411. For example, the sensors 1413 can be molded into the wearable structure 1411 or be integrated into a woven fabric (e.g., the sensors 1413 can be sewn into the fabric and mimic the pliability of fabric (e.g., the sensors 1413 can be constructed from a series of woven strands of fabric)).

The wearable structure 1411 can include flexible electronic connectors that interconnect the sensors 1413, the electronic circuitry, and/or other electronics components (described below in reference to FIG. 14B) that are enclosed in the wearable band 1410. In some embodiments, the flexible electronic connectors are configured to interconnect the sensors 1413, the electronic circuitry, and/or other electronics components of the wearable band 1410 with respective sensors and/or other electronics components of another electronic device (e.g., watch body 1420). The flexible electronic connectors are configured to move with the wearable structure 1411 such that the user adjustment to the wearable structure 1411 (e.g., resizing, pulling, folding, etc.) does not stress or strain the electrical coupling of components of the wearable band 1410.

As described above, the wearable band 1410 is configured to be worn by a user. In particular, the wearable band 1410 can be shaped or otherwise manipulated to be worn by a user. For example, the wearable band 1410 can be shaped to have a substantially circular shape such that it can be configured to be worn on the user's lower arm or wrist. Alternatively, the wearable band 1410 can be shaped to be worn on another body part of the user, such as the user's upper arm (e.g., around a bicep), forearm, chest, legs, etc. The wearable band 1410 can include a retaining mechanism 1412 (e.g., a buckle, a hook and loop fastener, etc.) for securing the wearable band 1410 to the user's wrist or other body part. While the wearable band 1410 is worn by the user, the sensors 1413 sense data (referred to as sensor data) from the user's skin. In particular, the sensors 1413 of the wearable band 1410 obtain (e.g., sense and record) neuromuscular signals.

The sensed data (e.g., sensed neuromuscular signals) can be used to detect and/or determine the user's intention to perform certain motor actions. In particular, the sensors 1413 sense and record neuromuscular signals from the user as the user performs muscular activations (e.g., movements, gestures, etc.). The detected and/or determined motor actions (e.g., phalange (or digits) movements, wrist movements, hand movements, and/or other muscle intentions) can be used to determine control commands or control information (instructions to perform certain commands after the data is sensed) for causing a computing device to perform one or more input commands. For example, the sensed neuromuscular signals can be used to control certain user interfaces displayed on the display 1405 of the wrist-wearable device 1400 and/or can be transmitted to a device responsible for rendering an artificial-reality environment (e.g., a head-mounted display) to perform an action in an associated artificial-reality environment, such as to control the motion of a virtual device displayed to the user. The muscular activations performed by the user can include static gestures, such as placing the user's hand palm down on a table; dynamic gestures, such as grasping a physical or virtual object; and covert gestures that are imperceptible to another person, such as slightly tensing a joint by co-contracting opposing muscles or using sub-muscular activations. The muscular activations performed by the user can include symbolic gestures (e.g., gestures mapped to other gestures, interactions, or commands, for example, based on a gesture vocabulary that specifies the mapping of gestures to commands).

The sensor data sensed by the sensors 1413 can be used to provide a user with an enhanced interaction with a physical object (e.g., devices communicatively coupled with the wearable band 1410) and/or a virtual object in an artificial-reality application generated by an artificial-reality system (e.g., user interface objects presented on the display 1405 or another computing device (e.g., a smartphone)).

In some embodiments, the wearable band 1410 includes one or more haptic devices 1446 (FIG. 14B; e.g., a vibratory haptic actuator) that are configured to provide haptic feedback (e.g., a cutaneous and/or kinesthetic sensation, etc.) to the user's skin. The sensors 1413, and/or the haptic devices 1446 can be configured to operate in conjunction with multiple applications including, without limitation, health monitoring, social media, games, and artificial reality (e.g., the applications associated with artificial reality).

The wearable band 1410 can also include coupling mechanism 1416 (e.g., a cradle or a shape of the coupling mechanism can correspond to shape of the watch body 1420 of the wrist-wearable device 1400) for detachably coupling a capsule (e.g., a computing unit) or watch body 1420 (via a coupling surface of the watch body 1420) to the wearable band 1410. In particular, the coupling mechanism 1416 can be configured to receive a coupling surface proximate to the bottom side of the watch body 1420 (e.g., a side opposite to a front side of the watch body 1420 where the display 1405 is located), such that a user can push the watch body 1420 downward into the coupling mechanism 1416 to attach the watch body 1420 to the coupling mechanism 1416. In some embodiments, the coupling mechanism 1416 can be configured to receive a top side of the watch body 1420 (e.g., a side proximate to the front side of the watch body 1420 where the display 1405 is located) that is pushed upward into the cradle, as opposed to being pushed downward into the coupling mechanism 1416. In some embodiments, the coupling mechanism 1416 is an integrated component of the wearable band 1410 such that the wearable band 1410 and the coupling mechanism 1416 are a single unitary structure. In some embodiments, the coupling mechanism 1416 is a type of frame or shell that allows the watch body 1420 coupling surface to be retained within or on the wearable band 1410 coupling mechanism 1416 (e.g., a cradle, a tracker band, a support base, a clasp, etc.).

The coupling mechanism 1416 can allow for the watch body 1420 to be detachably coupled to the wearable band 1410 through a friction fit, magnetic coupling, a rotation-based connector, a shear-pin coupler, a retention spring, one or more magnets, a clip, a pin shaft, a hook and loop fastener, or a combination thereof. A user can perform any type of motion to couple the watch body 1420 to the wearable band 1410 and to decouple the watch body 1420 from the wearable band 1410. For example, a user can twist, slide, turn, push, pull, or rotate the watch body 1420 relative to the wearable band 1410, or a combination thereof, to attach the watch body 1420 to the wearable band 1410 and to detach the watch body 1420 from the wearable band 1410. Alternatively, as discussed below, in some embodiments, the watch body 1420 can be decoupled from the wearable band 1410 by actuation of the release mechanism 1429.

The wearable band 1410 can be coupled with a watch body 1420 to increase the functionality of the wearable band 1410 (e.g., converting the wearable band 1410 into a wrist-wearable device 1400, adding an additional computing unit and/or battery to increase computational resources and/or a battery life of the wearable band 1410, adding additional sensors to improve sensed data, etc.). As described above, the wearable band 1410 (and the coupling mechanism 1416) is configured to operate independently (e.g., execute functions independently) from watch body 1420. For example, the coupling mechanism 1416 can include one or more sensors 1413 that contact a user's skin when the wearable band 1410 is worn by the user and provide sensor data for determining control commands.

A user can detach the watch body 1420 (or capsule) from the wearable band 1410 in order to reduce the encumbrance of the wrist-wearable device 1400 to the user. For embodiments in which the watch body 1420 is removable, the watch body 1420 can be referred to as a removable structure, such that in these embodiments the wrist-wearable device 1400 includes a wearable portion (e.g., the wearable band 1410) and a removable structure (the watch body 1420).

Turning to the watch body 1420, the watch body 1420 can have a substantially rectangular or circular shape. The watch body 1420 is configured to be worn by the user on their wrist or on another body part. More specifically, the watch body 1420 is sized to be easily carried by the user, attached on a portion of the user's clothing, and/or coupled to the wearable band 1410 (forming the wrist-wearable device 1400). As described above, the watch body 1420 can have a shape corresponding to the coupling mechanism 1416 of the wearable band 1410. In some embodiments, the watch body 1420 includes a single release mechanism 1429 or multiple release mechanisms (e.g., two release mechanisms 1429 positioned on opposing sides of the watch body 1420, such as spring-loaded buttons) for decoupling the watch body 1420 and the wearable band 1410. The release mechanism 1429 can include, without limitation, a button, a knob, a plunger, a handle, a lever, a fastener, a clasp, a dial, a latch, or a combination thereof.

A user can actuate the release mechanism 1429 by pushing, turning, lifting, depressing, shifting, or performing other actions on the release mechanism 1429. Actuation of the release mechanism 1429 can release (e.g., decouple) the watch body 1420 from the coupling mechanism 1416 of the wearable band 1410, allowing the user to use the watch body 1420 independently from wearable band 1410, and vice versa. For example, decoupling the watch body 1420 from the wearable band 1410 can allow the user to capture images using rear-facing camera 1425B. Although the coupling mechanism 1416 is shown positioned at a corner of watch body 1420, the release mechanism 1429 can be positioned anywhere on watch body 1420 that is convenient for the user to actuate. In addition, in some embodiments, the wearable band 1410 can also include a respective release mechanism for decoupling the watch body 1420 from the coupling mechanism 1416. In some embodiments, the release mechanism 1429 is optional and the watch body 1420 can be decoupled from the coupling mechanism 1416 as described above (e.g., via twisting, rotating, etc.).

The watch body 1420 can include one or more peripheral buttons 1423 and 1427 for performing various operations at the watch body 1420. For example, the peripheral buttons 1423 and 1427 can be used to turn on or wake (e.g., transition from a sleep state to an active state) the display 1405, unlock the watch body 1420, increase or decrease a volume, increase or decrease brightness, interact with one or more applications, interact with one or more user interfaces, etc. Additionally, or alternatively, in some embodiments, the display 1405 operates as a touch screen and allows the user to provide one or more inputs for interacting with the watch body 1420.

In some embodiments, the watch body 1420 includes one or more sensors 1421. The sensors 1421 of the watch body 1420 can be the same or distinct from the sensors 1413 of the wearable band 1410. The sensors 1421 of the watch body 1420 can be distributed on an inside and/or an outside surface of the watch body 1420. In some embodiments, the sensors 1421 are configured to contact a user's skin when the watch body 1420 is worn by the user. For example, the sensors 1421 can be placed on the bottom side of the watch body 1420 and the coupling mechanism 1416 can be a cradle with an opening that allows the bottom side of the watch body 1420 to directly contact the user's skin. Alternatively, in some embodiments, the watch body 1420 does not include sensors that are configured to contact the user's skin (e.g., including sensors internal and/or external to the watch body 1420 that configured to sense data of the watch body 1420 and the watch body 1420's surrounding environment). In some embodiments, the sensors 1413 are configured to track a position and/or motion of the watch body 1420.

The watch body 1420 and the wearable band 1410 can share data using a wired communication method (e.g., a Universal Asynchronous Receiver/Transmitter (UART), a USB transceiver, etc.) and/or a wireless communication method (e.g., near field communication, Bluetooth, etc.). For example, the watch body 1420 and the wearable band 1410 can share data sensed by the sensors 1413 and 1421, as well as application- and device-specific information (e.g., active and/or available applications), output devices (e.g., display, speakers, etc.), input devices (e.g., touch screen, microphone, imaging sensors, etc.).

In some embodiments, the watch body 1420 can include, without limitation, a front-facing camera 1425A and/or a rear-facing camera 1425B, sensors 1421 (e.g., a biometric sensor, an IMU sensor, a heart rate sensor, a saturated oxygen sensor, a neuromuscular signal sensor, an altimeter sensor, a temperature sensor, a bioimpedance sensor, a pedometer sensor, an optical sensor (e.g., imaging sensor 1463; FIG. 14B), a touch sensor, a sweat sensor, etc.). In some embodiments, the watch body 1420 can include one or more haptic devices 1476 (FIG. 14B; a vibratory haptic actuator) that is configured to provide haptic feedback (e.g., a cutaneous and/or kinesthetic sensation, etc.) to the user. The sensors 1421 and/or the haptic device 1476 can also be configured to operate in conjunction with multiple applications including, without limitation, health-monitoring applications, social media applications, game applications, and artificial-reality applications (e.g., the applications associated with artificial reality).

As described above, the watch body 1420 and the wearable band 1410, when coupled, can form the wrist-wearable device 1400. When coupled, the watch body 1420 and wearable band 1410 operate as a single device to execute functions (operations, detections, communications, etc.) described herein. In some embodiments, each device is provided with particular instructions for performing the one or more operations of the wrist-wearable device 1400. For example, in accordance with a determination that the watch body 1420 does not include neuromuscular signal sensors, the wearable band 1410 can include alternative instructions for performing associated instructions (e.g., providing sensed neuromuscular signal data to the watch body 1420 via a different electronic device). Operations of the wrist-wearable device 1400 can be performed by the watch body 1420 alone or in conjunction with the wearable band 1410 (e.g., via respective processors and/or hardware components) and vice versa. In some embodiments, operations of the wrist-wearable device 1400, the watch body 1420, and/or the wearable band 1410 can be performed in conjunction with one or more processors and/or hardware components of another communicatively coupled device (e.g., the HIPD 1370).

As described below with reference to the block diagram of FIG. 14B, the wearable band 1410 and/or the watch body 1420 can each include independent resources required to independently execute functions. For example, the wearable band 1410 and/or the watch body 1420 can each include a power source (e.g., a battery), a memory, data storage, a processor (e.g., a central processing unit (CPU)), communications, a light source, and/or input/output devices.

FIG. 14B shows block diagrams of a computing system 1430 corresponding to the wearable band 1410, and a computing system 1460 corresponding to the watch body 1420, according to some embodiments. A computing system of the wrist-wearable device 1400 includes a combination of components of the wearable band computing system 1430 and the watch body computing system 1460, in accordance with some embodiments.

The watch body 1420 and/or the wearable band 1410 can include one or more components shown in watch body computing system 1460. In some embodiments, a single integrated circuit includes all or a substantial portion of the components of the watch body computing system 1460 are included in a single integrated circuit. Alternatively, in some embodiments, components of the watch body computing system 1460 are included in a plurality of integrated circuits that are communicatively coupled. In some embodiments, the watch body computing system 1460 is configured to couple (e.g., via a wired or wireless connection) with the wearable band computing system 1430, which allows the computing systems to share components, distribute tasks, and/or perform other operations described herein (individually or as a single device).

The watch body computing system 1460 can include one or more processors 1479, a controller 1477, a peripherals interface 1461, a power system 1495, and memory (e.g., a memory 1480), each of which are defined above and described in more detail below.

The power system 1495 can include a charger input 1496, a power-management integrated circuit (PMIC) 1497, and a battery 1498, each are which are defined above. In some embodiments, a watch body 1420 and a wearable band 1410 can have respective charger inputs (e.g., charger input 1496 and 1457), respective batteries (e.g., battery 1498 and 1459), and can share power with each other (e.g., the watch body 1420 can power and/or charge the wearable band 1410, and vice versa). Although watch body 1420 and/or the wearable band 1410 can include respective charger inputs, a single charger input can charge both devices when coupled. The watch body 1420 and the wearable band 1410 can receive a charge using a variety of techniques. In some embodiments, the watch body 1420 and the wearable band 1410 can use a wired charging assembly (e.g., power cords) to receive the charge. Alternatively, or in addition, the watch body 1420 and/or the wearable band 1410 can be configured for wireless charging. For example, a portable charging device can be designed to mate with a portion of watch body 1420 and/or wearable band 1410 and wirelessly deliver usable power to a battery of watch body 1420 and/or wearable band 1410. The watch body 1420 and the wearable band 1410 can have independent power systems (e.g., power system 1495 and 1456) to enable each to operate independently. The watch body 1420 and wearable band 1410 can also share power (e.g., one can charge the other) via respective PMICs (e.g., PMICs 1497 and 1458) that can share power over power and ground conductors and/or over wireless charging antennas.

In some embodiments, the peripherals interface 1461 can include one or more sensors 1421, many of which listed below are defined above. The sensors 1421 can include one or more coupling sensors 1462 for detecting when the watch body 1420 is coupled with another electronic device (e.g., a wearable band 1410). The sensors 1421 can include imaging sensors 1463 (one or more of the cameras 1425 and/or separate imaging sensors 1463 (e.g., thermal-imaging sensors)). In some embodiments, the sensors 1421 include one or more SpO2 sensors 1464. In some embodiments, the sensors 1421 include one or more biopotential-signal sensors (e.g., EMG sensors 1465, which may be disposed on a user-facing portion of the watch body 1420 and/or the wearable band 1410). In some embodiments, the sensors 1421 include one or more capacitive sensors 1466. In some embodiments, the sensors 1421 include one or more heart rate sensors 1467. In some embodiments, the sensors 1421 include one or more IMUs 1468. In some embodiments, one or more IMUs 1468 can be configured to detect movement of a user's hand or other location that the watch body 1420 is placed or held.

In some embodiments, the peripherals interface 1461 includes an NFC component 1469, a global-position system (GPS) component 1470, a long-term evolution (LTE) component 1471, and/or a Wi-Fi and/or Bluetooth communication component 1472. In some embodiments, the peripherals interface 1461 includes one or more buttons 1473 (e.g., the peripheral buttons 1423 and 1427 in FIG. 14A), which, when selected by a user, cause operations to be performed at the watch body 1420. In some embodiments, the peripherals interface 1461 includes one or more indicators, such as a light emitting diode (LED), to provide a user with visual indicators (e.g., message received, low battery, an active microphone, and/or a camera, etc.).

The watch body 1420 can include at least one display 1405 for displaying visual representations of information or data to the user, including user-interface elements and/or three-dimensional (3D) virtual objects. The display can also include a touch screen for inputting user inputs, such as touch gestures, swipe gestures, and the like. The watch body 1420 can include at least one speaker 1474 and at least one microphone 1475 for providing audio signals to the user and receiving audio input from the user. The user can provide user inputs through the microphone 1475 and can also receive audio output from the speaker 1474 as part of a haptic event provided by the haptic controller 1478. The watch body 1420 can include at least one camera 1425, including a front-facing camera 1425A and a rear-facing camera 1425B. The cameras 1425 can include ultra-wide-angle cameras, wide-angle cameras, fish-eye cameras, spherical cameras, telephoto cameras, a depth-sensing cameras, or other types of cameras.

The watch body computing system 1460 can include one or more haptic controllers 1478 and associated componentry (e.g., haptic devices 1476) for providing haptic events at the watch body 1420 (e.g., a vibrating sensation or audio output in response to an event at the watch body 1420). The haptic controllers 1478 can communicate with one or more haptic devices 1476, such as electroacoustic devices, including a speaker of the one or more speakers 1474 and/or other audio components and/or electromechanical devices that convert energy into linear motion such as a motor, solenoid, electroactive polymer, piezoelectric actuator, electrostatic actuator, or other tactile output generating component (e.g., a component that converts electrical signals into tactile outputs on the device). The haptic controller 1478 can provide haptic events to respective haptic actuators that are capable of being sensed by a user of the watch body 1420. In some embodiments, the one or more haptic controllers 1478 can receive input signals from an application of the applications 1482.

In some embodiments, the computer system 1430 and/or the computer system 1460 can include memory 1480, which can be controlled by a memory controller of the one or more controllers 1477 and/or one or more processors 1479. In some embodiments, software components stored in the memory 1480 include one or more applications 1482 configured to perform operations at the watch body 1420. In some embodiments, the one or more applications 1482 include games, word processors, messaging applications, calling applications, web browsers, social media applications, media streaming applications, financial applications, calendars, clocks, etc. In some embodiments, software components stored in the memory 1480 include one or more communication interface modules 1483 as defined above. In some embodiments, software components stored in the memory 1480 include one or more graphics modules 1484 for rendering, encoding, and/or decoding audio and/or visual data; and one or more data management modules 1485 for collecting, organizing, and/or providing access to the data 1487 stored in memory 1480. In some embodiments, one or more of applications 1482 and/or one or more modules can work in conjunction with one another to perform various tasks at the watch body 1420.

In some embodiments, software components stored in the memory 1480 can include one or more operating systems 1481 (e.g., a Linux-based operating system, an Android operating system, etc.). The memory 1480 can also include data 1487. The data 1487 can include profile data 1488A, sensor data 1489A, media content data 1490, and application data 1491.

It should be appreciated that the watch body computing system 1460 is an example of a computing system within the watch body 1420, and that the watch body 1420 can have more or fewer components than shown in the watch body computing system 1460, combine two or more components, and/or have a different configuration and/or arrangement of the components. The various components shown in watch body computing system 1460 are implemented in hardware, software, firmware, or a combination thereof, including one or more signal processing and/or application-specific integrated circuits.

Turning to the wearable band computing system 1430, one or more components that can be included in the wearable band 1410 are shown. The wearable band computing system 1430 can include more or fewer components than shown in the watch body computing system 1460, combine two or more components, and/or have a different configuration and/or arrangement of some or all of the components. In some embodiments, all, or a substantial portion of the components of the wearable band computing system 1430 are included in a single integrated circuit. Alternatively, in some embodiments, components of the wearable band computing system 1430 are included in a plurality of integrated circuits that are communicatively coupled. As described above, in some embodiments, the wearable band computing system 1430 is configured to couple (e.g., via a wired or wireless connection) with the watch body computing system 1460, which allows the computing systems to share components, distribute tasks, and/or perform other operations described herein (individually or as a single device).

The wearable band computing system 1430, similar to the watch body computing system 1460, can include one or more processors 1449, one or more controllers 1447 (including one or more haptics controller 1448), a peripherals interface 1431 that can include one or more sensors 1413 and other peripheral devices, power source (e.g., a power system 1456), and memory (e.g., a memory 1450) that includes an operating system (e.g., an operating system 1451), data (e.g., data 1454 including profile data 1488B, sensor data 1489B, etc.), and one or more modules (e.g., a communications interface module 1452, a data management module 1453, etc.).

The one or more sensors 1413 can be analogous to sensors 1421 of the computer system 1460 in light of the definitions above. For example, sensors 1413 can include one or more coupling sensors 1432, one or more SpO2 sensors 1434, one or more EMG sensors 1435, one or more capacitive sensors 1436, one or more heart rate sensors 1437, and one or more IMU sensors 1442.

The peripherals interface 1431 can also include other components analogous to those included in the peripheral interface 1461 of the computer system 1460, including an NFC component 1439, a GPS component 1440, an LTE component 1451, a Wi-Fi and/or Bluetooth communication component 1442, and/or one or more haptic devices 1476 as described above in reference to peripherals interface 1461. In some embodiments, the peripherals interface 1431 includes one or more buttons 1443, a display 1433, a speaker 1444, a microphone 1445, and a camera 1455. In some embodiments, the peripherals interface 1431 includes one or more indicators, such as an LED.

It should be appreciated that the wearable band computing system 1430 is an example of a computing system within the wearable band 1410, and that the wearable band 1410 can have more or fewer components than shown in the wearable band computing system 1430, combine two or more components, and/or have a different configuration and/or arrangement of the components. The various components shown in wearable band computing system 1430 can be implemented in one or a combination of hardware, software, and firmware, including one or more signal processing and/or application-specific integrated circuits.

The wrist-wearable device 1400 with respect to FIG. 14A is an example of the wearable band 1410 and the watch body 1420 coupled, so the wrist-wearable device 1400 will be understood to include the components shown and described for the wearable band computing system 1430 and the watch body computing system 1460. In some embodiments, wrist-wearable device 1400 has a split architecture (e.g., a split mechanical architecture or a split electrical architecture) between the watch body 1420 and the wearable band 1410. In other words, all of the components shown in the wearable band computing system 1430 and the watch body computing system 1460 can be housed or otherwise disposed in a combined watch device 1400, or within individual components of the watch body 1420, wearable band 1410, and/or portions thereof (e.g., a coupling mechanism 1416 of the wearable band 1410).

The techniques described above can be used with any device for sensing neuromuscular signals, including the arm-wearable devices of FIG. 14A-14B, but could also be used with other types of wearable devices for sensing neuromuscular signals (such as body-wearable or head-wearable devices that might have neuromuscular sensors closer to the brain or spinal column).

In some embodiments, a wrist-wearable device 1400 can be used in conjunction with a head-wearable device (e.g., AR device 1360 and VR device) and/or an HIPD 1370, and the wrist-wearable device 1400 can also be configured to be used to allow a user to control aspect of the artificial reality (e.g., by using EMG-based gestures to control user interface objects in the artificial reality and/or by allowing a user to interact with the touchscreen on the wrist-wearable device to also control aspects of the artificial reality).

Any data collection performed by the devices described herein and/or any devices configured to perform or cause the performance of the different embodiments described above in reference to any of the Figures, hereinafter the “devices,” is done with user consent and in a manner that is consistent with all applicable privacy laws. Users are given options to allow the devices to collect data, as well as the option to limit or deny collection of data by the devices. A user is able to opt-in or opt-out of any data collection at any time. Further, users are given the option to request the removal of any collected data.

It will be understood that, although the terms “first,” “second,” etc. may be used herein to describe various elements, these elements should not be limited by these terms. These terms are only used to distinguish one element from another.

The terminology used herein is for the purpose of describing particular embodiments only and is not intended to be limiting of the claims. As used in the description of the embodiments and the appended claims, the singular forms “a,” “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” can be construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” can be construed to mean “upon determining” or “in response to determining” or “in accordance with a determination” or “upon detecting” or “in response to detecting” that the stated condition precedent is true, depending on the context.

The foregoing description, for purpose of explanation, has been described with reference to specific embodiments. However, the illustrative discussions above are not intended to be exhaustive or to limit the claims to the precise forms disclosed. Many modifications and variations are possible in view of the above teachings. The embodiments were chosen and described in order to best explain principles of operation and practical applications, to thereby enable others skilled in the art.

What is claimed is:

1. A wearable structure, comprising:
a flexible printed circuit (FPC) layer within the wearable structure, the FPC layer configured to couple with one or more biopotential signal-processing components along a coupling length of the FPC layer; and
a strain-relief layer coupled with a portion of the FPC layer such that the strain-relief layer spans the coupling length of the FPC layer,
wherein a strain applied to the wearable structure is relieved by the strain-relief layer such that the strain is not transferred to the one or more biopotential signal-processing components.

2. The wearable structure of claim 1, wherein a twisting force applied to the wearable structure is relieved by the strain-relief layer such that the twisting force is not transferred to the one or more biopotential signal-processing components.

3. The wearable structure of claim 1, wherein the strain-relief layer is stretch resistant such that the FPC layer is not stretched when a tensile force is applied to the wearable structure.

4. The wearable structure of claim 1, wherein the one or more biopotential signal-processing components are electrically coupled with one or more biopotential-signal-sensing electrodes.

5. The wearable structure of claim 1, wherein the FPC layer and the strain-relief layer are part of a FPC assembly.

6. The wearable structure of claim 1, wherein the strain-relief layer is coupled to a bottom surface of the FPC layer.

7. The wearable structure of claim 1, wherein the strain-relief layer is coupled between traces of the FPC layer.

8. The wearable structure of claim 1, wherein the strain-relief layer reduces or eliminates neutral axis deflection of the FPC layer.

9. The wearable structure of claim 1, wherein the strain-relief layer has a first length and the FPC layer has a second length, the first length being greater than the second length.

10. The wearable structure of claim 1, wherein the strain-relief layer does not stretch.

11. The wearable structure of claim 1, wherein the strain-relief layer is formed of Vectran, Kevlar, or other polymers.

12. The wearable structure of claim 1, further comprising:
a metal layer coupled to the strain-relief layer, the metal layer configured to support the FPC layer and the strain-relief layer.

13. The wearable structure of claim 1, further comprising:
an outer layer embedding at least the FPC layer, the strain-relief layer, and the one or more biopotential signal-processing components.

14. The wearable structure of claim 1, wherein the wearable structure is at least one of: a wrist-wearable device, head-wearable device, or wearable garment.

15. The wearable structure of claim 1, wherein the strain-relief layer has a predetermined thickness.

16. The wearable structure of claim 1, wherein:
the wearable structure is configured to couple with a compute core; and
the compute core is configured to communicatively couple with the FPC layer.

17. A wearable electronic device, comprising:
one or more biopotential signal-processing components; and
a wearable structure, including:
a flexible printed circuit (FPC) layer within the wearable structure, the FPC layer configured to couple with the one or more biopotential signal-processing components along a coupling length of the FPC layer; and
a strain-relief layer coupled with a portion of the FPC layer such that the strain-relief layer spans the coupling length of the FPC layer,
wherein a strain applied to the wearable structure is relieved by the strain-relief layer such that the strain is not transferred to the one or more biopotential signal-processing components.

18. The wearable electronic device of claim 17, wherein a twisting force applied to the wearable structure is relieved by the strain-relief layer such that the twisting force is not transferred to the one or more biopotential signal-processing components.

19. A method of manufacturing a wearable structure, comprising:
providing a wearable structure that comprises a flexible printed circuit (FPC) and a strain-relief layer, wherein:
the FPC is within the wearable structure and configured to couple with biopotential signal-processing components along a coupling length of the FPC layer;
the strain-relief layer coupled with a portion of the FPC layer such that the strain-relief layer spans the coupling length of the FPC layer,
wherein a strain applied to the wearable structure is relieved by the strain-relief layer such that the strain is not transferred to the one or more biopotential signal-processing components.

20. The method of manufacturing the wearable structure of claim 19, wherein a twisting force applied to the wearable structure is relieved by the strain-relief layer such that the twisting force is not transferred to the one or more biopotential signal-processing components.
